(19) United States
(12) Patent Application Publication
(10) Pub. No.: US 2003/0166139 A1
(43) Pub. Date: Sep. 4, 2003

(54) Rotavirus VP6 subunit

(76) Inventors: Anthony H. Choi, Park Hills, KY (US); Richard L. Ward, Cincinnati, OH (US)

Correspondence Address: Loy M. White, Cincinnati Children's Hospital Medical Center, Office of Intellectual Property & Venture Dev't., 3333 Burnet Avenue, MLC 7032, Cincinnati, OH, 45229-3039, US

(21) Appl. No.: 10/200,381

(22) Filed: Jul. 22, 2002

Related U.S. Application Data
(60) Provisional application No. 60/306,763, filed on Jul. 20, 2001.

(51) Int. Cl. C07K 14/14; A61K 39/12; A61K 39/15; C07H 21/04; A61K 45/00; C07K 14/00; C12P 21/06; C12N 15/09; A61K 39/00; C07K 1/00; C07K 17/00

(52) U.S. Cl. 435/69.1; 424/215.1; 424/186.1; 530/350; 424/185.1; 424/192.1; 424/282.1; 530/826; 536/23.72; 435/69.3

(57) ABSTRACT

The present invention relates to vaccine compositions comprising the VP6 protein from mouse (EDIM) and human (CJN) rotavirus strains. Methods of making the described immunogenic VP6 proteins and methods of using the described compositions are also disclosed.

### BACKGROUND

Rotavirus is the most common cause of severe gastroenteritis worldwide in children less than 3 years of age. Diarrhea occurs by the triggering of the intestinal nervous system to secrete water excessively. Nausea and fever sometimes accompany diarrhea. These symptoms usually last a week. Over time, the epithelial lining repairs itself and normal digestion recovers quickly if the patient is well-hydrated.

In developing countries, rotavirus-induced dehydration causes 600,000 to 870,000 deaths each year, accounting for about 20 to 23% of all deaths due to diarrhea. In the United States, it accounts for approximately 500,000 physician visits and 50,000 hospitalization per year among children age <5 years and 20 to 125 deaths. Therefore, rotavirus causes both morbidity and mortality worldwide.

Vaccination continues to be the most viable control measure to have an impact on severe rotavirus disease. The World Health Organization (WHO) highly recommends the development and evaluation of rotavirus vaccines.

The first-generation rotavirus vaccines were live, orally administered rotavirus strains. One of these vaccines is an attenuated human rotavirus strain presently being developed by GlaxoSmithKline. Two other vaccines are based on reassortant animal strains composed of several rotaviruses, each of which have one neutralizing protein gene segment replaced by a human rotavirus segment. One of these reassortant vaccines, the FDA-approved Rhesus Rotavirus Reassortant Tetravalent Vaccine (RRV-TV), was developed and marketed by Wyeth Lederle Vaccines and Pediatrics Center for Disease Control and Prevention. The other reassortant vaccine is a bovine reassortant rotavirus vaccine developed by Merck Research Laboratories. The two reassortants vaccines were formulated to target common, circulating rotavirus G and P serotypes. G and P serotyping, is based on the ability of the outer capsid VP4 and VP7 proteins to independently induce neutralizing antibodies. The reassortant rotavirus vaccines are designed to target the most common circulating G and P serotypes but they may not be effective against emerging serotypes. The subject invention is to target all rotavirus strains through both humoral and cellular immune mechanisms.

A number of disadvantages are associated with live-oral rotavirus vaccines. First, the presence of maternal antibodies in infants can interfere with the take of the live-oral vaccines. Following primary immunization, antibodies, which are developed against the vaccines, can interfere with the take of the second and third immunizations. Second, a low-grade fever and occasionally, diarrhea and irritability had been reported following immunization with RRV-TV. Third, there is a possibility that the attenuated live-oral rotavirus vaccines revert to regain virulence and spread to other persons. Finally, live-oral vaccines are expensive and beyond the monetary means of most low-income countries.

In addition to the above disadvantages, the live-oral RRV-TV rotavirus vaccine has been associated with bowel blockage. The RRV-TV vaccine was evaluated in Finland, Venezuela, and the United States through studies involving approximately 18,000 infants. In the studies, three doses of the vaccine were given to infants aged 6 weeks to 26 weeks at the time of their first dose. The results showed about 50% protection against all rotavirus-induced diarrhea, but approximately 75% protection against severe rotavirus-induced diarrhea. Based upon these studies, the FDA approved RRV-TV in 1998 for immunization in the United States and within a year the vaccine had been administered to 1.5 million children. However, in 1999 the vaccine was withdrawn from the market due to reports of instussusception (bowel blockage) among some infants following rotavirus vaccination. The exact cause of the bowel obstruction is not known but it is believed to be related to virus replication in the intestine.

It has further been suggested that rotavirus infection and possibly live rotavirus vaccines may lead to or exacerbate childhood type I diabetes. The link between rotavirus infection and diabetes was based on a 6-year study of 54 babies who were at risk for the development of type I diabetes. This type of diabetes is an autoimmune disease that appears to involve CD4+ T cells and begins early in life. All of the 54 infants became infected with rotaviruses and 24 of these children showed clear signs of developing diabetes. The levels of autoantibodies, which are signs of an autoimmune attack of the pancreas, increased with each rotavirus infection. The mechanism by which rotavirus might trigger diabetes is unclear but it appears to involve rotavirus-mediated molecular mimicry. The peptide epitopes that appear to be responsible for molecular mimicry are mapped to the rotavirus VP7 protein.

As noted above, live oral rotavirus vaccines may, cause bowel obstruction and may be linked to childhood diabetes. In view of these and other negative characteristics associated with the currently available vaccines, a second generation of vaccines needs to be developed which will provide a safe and effective alternative to live-oral vaccines. Subunit vaccines are thought to be generally safer than live-attenuated or killed virus vaccines since only a portion of the virus is used to induce an immune response, as opposed to the entire virus. Presently, rotavirus subunit vaccines that include non-infectious virus-like particles (VLPs), which do not contain rotavirus genomes and are produced by recombinant baculoviruses in insect cells, may represent one possible avenue for the development of an improved vaccine.

The production of VLPs involves co-infection of insect cells with recombinant baculoviruses expressing the inner capsid VP2 or middle capsid VP6 proteins. In infected insect cells, VP2 and VP6 proteins assemble into double-layered VLPs (2/6 VLPs). VLPs (2/6/4/7 VLPs) with the outer capsid VP4 and VP7 proteins assembled on the 2/6-VLPs have also been constructed. These VLP vaccines have been tested by intranasal administration with an adjuvant in the mouse model and were found to provide at least some protection against rotavirus infection.

Other vaccines are still in development, including DNA vaccines and recombinant viruses or bacteria containing rotavirus genes. DNA vaccine technology entails the cloning of foreign genes into mammalian expression plasmids. Recombinant DNA plasmids have been delivered via various mucosal and parenteral routes, including skin immunization using the gene gun. Protection against experimental infection in a number of pathogen/animal models has been successfully demonstrated. Recently, clinical trials to evaluate DNA vaccines encoding, e.g. hepatitis B surface antigen or malaria antigens, have been carried out. DNA vaccines constructed from mouse rotavirus genes encoding VP4, VP6 or VP7 have also been evaluated. Mice were immunized with recombinant plasmids using the gene gun, as well as by intradernal, intranasal, and intraperitoneal immunization. The protective effect was measured after oral challenge with mouse EDIM rotavirus. None of the DNA vaccines, however, induced protection in the mouse model. In contrast, another laboratory reported partial protection against rotavirus infection in the adult mouse model using DNA vaccines. Because the methodologies used by the two laboratories were essentially identical, the discrepancies between the results obtained by these two laboratories cannot be readily explained. The inconsistencies in immunization outcomes raise doubts about the feasibility of this method of vaccination in humans. Therefore, recombinant DNA does not appear to be a suitable form of rotavirus vaccine.

Other laboratories have explored the use of live viral vectors (HSV and poliovirus) as potential vehicles for delivery of rotavirus genes. Similarly, bacteria (Salmonella and Shigella) have also been explored as potential vaccine vectors. However, the rotavirus proteins being expressed have been limited to VP4 and VP7, and no preclinical data are available on the efficacies of these vaccines.

The above review of the state of the art attempts to provide some measure of the extent of time and energy that has been expended to date to develop an effective rotavirus vaccine. Yet, even with the most successful efforts to date, rotavirus disease can be prevented only some of the time. Given this limitation, there remains a need for a safe and effective rotavirus vaccine which overcomes the above deficiencies.

While the use of entire viral proteins represents an advancement over whole, live virus vaccines, this approach could also be improved using partial proteins as subunit vaccines. Subunit vaccines are thought to be generally safer than live-attenuated or killed virus vaccines since only a portion of the virus is used to induce an immune response, as opposed to the entire virus. Such vaccines would also reduce the cost of preparation and decrease the difficulties in preparing the needed quantities of whole viral protein to be used in the vaccine preparations. The present invention is designed to remedy and eliminate these problems. The present invention relates to the development of a subunit rotavirus vaccine that is a safe and efficacious alternative to the current whole, live rotavirus vaccines.

### SUMMARY OF THE INVENTION

The present invention relates to the discovery that the inner capsid VP6 protein, preferably when formulated with an adjuvant, induces greater than 99% protection from rotavirus infection in a mammal, and that the immunological characterization of VP6 proteins indicate the vaccine does not require B-cell, CD8⁺-cell functions, and/or VP6-specific antibodies. The VP6 proteins were derived from mouse (EDI) and human (CJN) rotavirus strains, and have been recombinantly produced, and preferably formulated with an adjuvant, in novel subunit vaccines for providing protection from rotavirus infection in mammalian subjects.

Accordingly, in one embodiment, the subject invention is directed to isolation of nucleic acid molecules comprising the native, unmodified coding sequence for the immunogenic VP6 protein of the human CJN rotavirus strain and the mouse rotavirus strain, or a fragment of the nucleic acid molecules comprising at least 15 nucleotides.

In an additional embodiment, the subject invention is directed to synthetic, codon-optimized nucleic acid molecules assembled from oligonucleotides comprising the coding sequence for the immunogenic VP6 protein of the human CJN rotavirus strain, or a fragment of the nucleic acid molecule comprising at least 15 nucleotides.

In a still additional embodiment, the invention is directed to recombinant plasmids including nucleic acid molecules encoding the immunogenic VP6 protein of the rotavirus strains described herein, host cells transformed with these plasmids, and methods of producing recombinant rotavirus VP6 proteins.

In a still further embodiment, the subject invention is directed to vaccine compositions comprising a pharmaceutically acceptable vehicle, an immunogenic VP6 protein, e.g., a rotavirus VP6 protein or an immunogenic fragment of rotavirus VP6 protein comprising at least 5 amino acids, and an adjuvant, as well as methods of preparing the vaccine compositions.

In yet another embodiment, the present invention is directed to methods of preventing rotavirus infections in a mammal. The method comprises administering to the mammal a prophylactically effective amount of the above vaccine compositions.

These and other embodiments of the present invention will be readily apparent to those of ordinary skill in the art in view of the disclosure herein.

### BRIEF DESCRIPTION OF THE DRAWINGS

FIG. 1 is a schematic of the pMAL/c2X plasmid. This plasmid is used in cloning the gene sequence encoding rotavirus VP6 proteins. It encodes the chimeric MBP::LacZα protein which consists of the genetically fused maltose-binding protein and the LacZα peptide. The multiple cloning sites (MCS) are shown with the available restriction sites. Insertion of a nucleotide sequence encoding for VP6 which contains a stop codon results in ultimate expression of chimeric MBP::VP6 proteins.

FIG. 2 is an immunoblot of chimeric MBP::EDIM-VP6 containing the fusion partner maltose-binding protein (MBP) purified from E. coli cell lysate. Cells were harvested 3 hours after addition of IPTG. The cellular proteins were separated by SDS-gel electrophoresis and subjected to immunoblot analysis. Lane 1 shows purified maltose-binding protein, lane 2 shows cell lysate, and lane 3 shows chimeric EDIM-VP6 purified using amylose affinity resin. The arrow points to putative full-length chimeric VP6. Numerous truncated VP6 proteins were also obtained. The truncated proteins lack various portions of their carboxyl terminals.

FIG. 3 is an immunoblot of chimeric MBP::EDIM-VP6::His6, containing the fusion partners maltose-binding protein (MBP) and His6, purified from E. coli cell lysates. Cells were harvested 3 hours after addition of IPTG. The cellular proteins were separated by SDS-gel electrophoresis and then subjected to immunoblot analysis. Lane 1 shows maltose-binding protein, lane 2 shows cell lysate, and lane 3 shows chimeric MBP::EDIM-VP6 purified using amylose and Talon affinity resins. Panel A shows anti-MBP as primary anti-serum and panel B shows anti-His6 as primary antibody. This method of sequential affinity resin binding allowed isolation of substantially pure full-length recombinant VP6 proteins. The arrows point to full-length chimeric VP6 proteins.

FIG. 4 is a sucrose gradient centrifugation analysis showing chimeric MBP::VP6 does not form virus-like particles (VLPs). Amylose resin purified chimeric EDIM VP6 (A) and purified EDIM rotavirus particles (B) were subjected to sucrose gradient centrifugation. Fractions were collected and analysed by (A) immunoblot analyses using anti-MBP antiserum as the primary antibody and (B) SDS-gel electrophoresis and silver staining. Rotavirus particles readily entered into the gradient (B. Fractions 11 and 12) and virus aggregates were pelleted in the bottom fraction (B. Fraction 16). In contrast, chimeric MBP::VP6 remained at the top of the gradient (A. Fractions 1 and 2). The results in this study have provided evidence that chimeric VP6 does not form structures with buoyant properties of rotavirus particles.

FIG. 5 shows the time course of shedding of rotavirus in stool samples. The time course of fecal shedding of rotavirus in mice that were not immunized or immunized with an EDIM-VP6-containing formulation and subsequently challenged with EDIM is shown. The open circles represent data points from EDIM challenged control mice. The squares represent data points from EDIM challenged experimental mice that were intranasally vaccinated with the VP6::MBP rotavirus fusion vaccine composition of the present invention. Also shown is the calculation using the amount of rotavirus antigen quantified from stool samples to determine the efficacy of vaccine composition containing the present invention. The composition of the present invention induced greater than 99% protection from shedding of rotavirus in the stool samples.

FIG. 6 is an immunoblot analysis showing induction of VP6-specific antiserum in mice immunized with the MBP::EDIM VP6. Proteins from purified rotavirus particles were used for analyses. Lane 1 shows the primary antiserum from control mice, lane 2 shows the anti-serum from mice immunized with the composition of the present invention containing MBP::VP6 and LT(R192G). The VP6 protein was recognized by the immunized serum. The arrow indicates that only VP6 was recognized by the specific-serum.

FIG. 7 shows the effects of either CD8 or CD4 T-cell depletion on shedding of rotavirus antigen in either naive or VP6-immunized, B-cell-deficient J_{H}D mice during the 7 days after EDIM challenge. Groups of six J_{H}D mice were either not immunized or immunized intranasally with two doses of MBP::VP6 and LT(R192G) separated by 2 weeks. Starting at 24 days after the second dose, some groups of mice were depleted of either CD8 or CD4 T cells by daily (4 consecutive days) injections with MAbs specific for each cell type. On day 28 after the second dose, all mice were challenged with 1,000 SD₅₀ of wild-type EDIM and monitored daily for shedding of rotavirus antigen during the following 7 days. Two additional MAb injections were administered during the 7-day analysis period. The results represent the average amounts in nanograms (ng) of rotavirus antigen shed/mouse/day during the 7-day period, with standard deviations shown by the error bars.

FIG. 8 shows the alignment of the amino acid sequence of the CJN and EDIM VP6 proteins. EDIM is a mouse strain and CJN is a human strain of rotavirus. They have 91% homology. The 35 amino acid differences between them are shown.

FIG. 9 is an immunoblot analysis of expressed chimeric MBP::CJN-VP6::His6. E. coli cells containing expressed MBP::CJN-VP6::His6 proteins were subjected to SDS-PAGE and immunoblot analysis. The blot was probed with the primary antibodies raised against (1) MBP (New England Biolabs), (2) human group A rotavirus (DAKO), and (3) His6 (Santa Cruz). The arrows point to the putative full-length VP6 proteins.

FIG. 10 is an immunoblot analyses of resin purified chimeric MBP::CJN-VP6::His6 protein. The proteins were sequentially purified using amylose resin and Talon resin. Lane 1 shows purified MBP, lane 2 shows protein samples before binding to resins, and lane 3 shows purified MBP::CJN-VP6::His6 after amylose and Talon resin purification. The primary antibodies used are anti-MBP (panel A) and anti-His6 (panel B). The arrows point to full-length VP6 proteins. This method of sequential affinity resin purification enabled the isolation of substantially full-length recombinant VP6 proteins. The arrows point to the full-length chimeric VP6 proteins.

FIG. 11 is a schematic of the plasmid pRARE present in Rosetta cells. The Rosetta cell strain contains a plasmid called pRARE which encodes rare tRNAs that are frequently used by overexpressed recombinant proteins. The plasmid encoding the native unmodified CJN VP6 gene was transformed into Rosetta cells. Expression of recombinant VP6 was induced by the addition of IPTG.

FIG. 12 is an immunoblot showing enhanced expression of MBP::CJN-VP6::His6. The plasmid expressing native unmodified CJN VP6 or expressing the codon-optimized synthetic VP6 was transformed into BL21 or Rosetta E. coli cells. Following IPTG induction the cells were harvest at 3 hours post-induction. The proteins were separated by SDS-gel and then subjected to immunoblot analyses. The primary anti-sera used were (A) anti-MBP and (B) anti-His6. Lane 1 shows VP6 expressed from the native unmodified gene in BL21 cells, lane 2 shows VP6 expressed from the native unmodified gene in Rosetta cells, lane 3 shows VP6 expressed from the codon-optimized gene in BL21 cells, and lane 4 shows VP6 expressed from the codon-optimized gene in Rosetta cells.

### DETAILED DESCRIPTION OF THE INVENTION

As used herein, the phrase “substantially the same” or “substantially identical” to describe the identity of polynucleotides, means a nucleic acid or polynucleotide exhibiting at least 80%, preferably at least 85%, more preferably at least 90%, and most preferably at least 95% homology to a reference nucleic acid. For nucleotide sequences, the length of comparison sequences will generally be at least 10 to 500 nucleotides in length. More specifically, the length of comparison will be at least 50 nucleotides, preferably at least 60 nucleotides, more preferably at least 75 nucleotides, and most preferably at least 110 nucleotides in length.

As used herein, the phrase “substantially the same” or “substantially identical” may also be applied to compare sequence similarity and identity of polypeptides and means a polypeptide exhibiting at least 80%, preferably at least 85%, more preferably at least 90%, and most preferably at least 95% homology to a reference amino acid sequence. For polypeptides, the length of comparison sequences will generally be at least 16 amino acids, preferably at least 20 amino acids, more preferably at least 25 amino acids, and most preferably at least 35 amino acids.

As used herein, the phrase “functional fragments” means those fragments of SEQ. ID. NO.: 2 and SEQ. ID. NO. 4, or other proteins that have a similar amino acid sequence as that of the CJN VP6 protein, that is capable of inducing an immune response from a subject upon exposure thereto. One of skill in the art can screen for the functionality of a fragment by using the examples provided herein, where a full-length VP6 protein is described. It is also envisioned that fragments of the VP6 protein can be identified in a similar manner. The phrase “substantially identical” means an amino acid sequence which differs only by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein assayed, (e.g., as described herein). Preferably, such a sequence is at least 85%, and more preferably from 90% to 100% homologous at the amino acid level to SEQ. ID. NO.: 2 and SEQ. ID. NO. 4.

As used herein, the phrase “substantially pure polypeptide” means a VP6 protein that has been separated from components that naturally accompany it. Typically, the polypeptide is substantially pure when it is at least 60%, by weight, free from the proteins and other naturally occurring molecules with which it is typically associated. Preferably, the preparation is at least 75%, more preferably at least 90%, and most preferably at least 99%, by weight, VP6 protein. A substantially pure VP6 polypeptide can be obtained, for example, by extraction from a natural source, by expression of a recombinant nucleic acid encoding a VP6 polypeptide, or by chemically synthesizing the protein. Purity can be measured by any appropriate method, e.g., column chromatography, polyacrylamide gel electrophoresis, or by HPLC analysis.

As used herein, a protein is “substantially free of naturally associated components” when it is separated from those contaminants that accompany it in its natural state. Thus, a protein that is chemically synthesized or produced in a cellular system different from the cell from which it naturally originates will be substantially free from its naturally associated components. Accordingly, substantially pure polypeptides include those derived from eukaryotic organisms but synthesized in E. coli or other prokaryotes.

As used herein, the phrase “synthetic gene” or “synthetic gene sequence” means a polynucleotide sequence containing an open reading frame that is chemically synthesized from individual nucleotides or from a series of oligonucleotides, whose size is evident to those knowledgeable of the art. The synthetic gene may contain additions, deletions or mutations to increase, decrease or impart no changes to the immunological and/or vaccine property of the protein. Additionally, the synthetic gene sequence may be “codon optimized” for enchanced production in transformed host organisms. Accordingly, the phrases “modified gene”, “modified, synthetic gene sequence”, or “codon-optimized synthetic gene” applies to the gene sequence when the gene sequence has been modified as described above.

As used herein, the phrase “native protein” means a protein that has not been modified, for example, by modification by genetic fusion with fusion partner polypeptide or fusion partner polypeptides. Accordingly, the phrase “fusion protein” means a protein of interest that is in genetic association with or chemically fused to user-selected polypeptides, whose function may be to provide polypeptides that facilitate affinity purification of the fusion protein and/or to provide adjuvant function. The phrase “in genetic association” means a contiguous sequence of amino acids produced from an mRNA produced from a gene containing codons for the amino acids of the rotavirus protein and the fusion protein partner. In one embodiment, a suitable fusion protein partner may consist of a protein that will either enhance or at least not diminish the recombinant expression of the rotavirus fusion protein product when the two are in genetic association. The phrase “chimeric”, when applied to fusion proteins, means proteins that are chimeras formed out of rotavirus proteins and fusion polypeptide partners.

As used herein, the term “adjuvants” means substances that, when incorporated into immunogenic compositions, act to accelerate, prolong, or enhance the quality of specific immune responses to the antigens contained therein. Adjuvants are considered to exert their effects through one or both of two mechanisms of action. One mechanism operates through their immunomodulating ability to quantitatively and qualitatively modify the immune response engendered to the vaccine antigens. The second mechanism involves the ability to physically present immunogenic composition components to the immune system. In this instance, the adjuvants associate with antigens physically retaining them in high concentrations. These “depots” then slowly release the trapped antigens. Adjuvant-delivery systems target vaccines to specific anatomical sites where they induce the immune responses against the intended pathogens. Examples of these delivery systems are compounds that can encapsulate vaccine proteins into microparticles, time-regulated delivery systems, and others.

Central to the present invention is the discovery that formulations of recombinantly produced rotavirus VP6 proteins and adjuvants have strong vaccine function delivered via mucosal routes. Also central to the present invention is the discovery that the disclosed invention does not require VP6-specific antibodies, CD8+ cells and B cells for vaccine efficacy. In particular, the genes for the VP6 proteins of the mouse EDIM strain and human CJN strains of rotavirus have been isolated, sequenced and characterized, and the protein sequences for the EDIM VP6 (SEQ. ID. NO. 2) and CJN VP6 (SEQ. ID. NO. 4) deduced therefrom. The complete DNA sequences of EDIM and CJN VP6 genes are disclosed (SEQ. ID. NO. 1 and SEQ. ID. NO. 3, respectively). Also central to the present invention is the design of a modified codon-optimized VP6 gene sequence (SEQ. ID. NO. 5) that contains codons favorably used by host organisms to enhance expression of recombinant VP6 protein used in vaccine formulations.

The description below relates to the generation of rotavirus subunit proteins for use in vaccine compositions. The description also relates to methods of providing protective immunity to vertebrates, including humans, against rotavirus infection or disease. In one embodiment, the immunogenic compositions comprise a human rotavirus VP6 protein and a fusion partner, which is preferably a molecule capable of functioning as an adjuvant. In one embodiment, the immunogenic composition comprise a human rotavirus VP6 protein, with the fusion partner removed, and an adjuvant. In another embodiment, the fusion protein comprises a rotavirus subunit amino acid sequence encoding the VP6 protein derived from the human group A CJN rotavirus. In a preferred embodiment, the VP6 protein in the composition comprises the rotavirus subunit amino acid sequence encoding the VP6 protein derived from the human CJN rotavirus and an adjuvant.

The native, recombinant, or fusion proteins of the present invention are composed of rotavirus VP6 protein or immunogenic fragment thereof. In an embodiment, the rotavirus protein used in the fusion protein construct is the VP6 protein or an immunogenic fragment thereof. In a preferred embodiment, the VP6 protein or an immunogenic fragment thereof may be used alone in the present invention. In a preferred embodiment, the VP6 amino acid sequence for human group A CJN rotavirus is used in the immunogenic compositions described below. The nucleotide sequence of the CJN VP6 gene is found at SEQ. ID. NO.: 3 and the corresponding amino acid sequence is found at SEQ. ID. NO.: 4.

Nucleotide sequences encoding the EDIM VP6 and CJN VP6 proteins disclosed herein (SEQ. ID. NO.: 1 and SEQ. ID. NO.3, respectively) can be used to identify and isolate polynucleotide molecules encoding VP6 proteins of other rotavirus strains using methods evident to those who are skilled in the art. Such techniques include, but are not limited to: 1) polymerase chain reaction (PCR) using genomic RNA, free cDNA or cDNA cloned in plasmid libraries, fecal samples of infected individuals, and primers capable of annealing to the DNA or RNA sequence of interest, 2) computer searches of sequence databases for similar sequences, and 3) antibody screening of expression libraries to detect cloned DNA with shared antigenic features.

The amplified gene sequences may be subjected to nucleotide sequencing for verification of the putative VP6 genes. The PCR conditions used for identifying and isolating VP6 sequences will be evident to one skilled in the art and will generally be guided by the purpose of the primer/template hybridization, the type of hybridization (DNA-DNA or DNA-RNA), and the level of desired relatedness between the sequences.

Vaccine compositions described herein can be formulated with polynucleotides having substantially the same nucleotide sequence set forth in SEQ. ID. NO.: 1 and SEQ. ID. NO.: 3, or functional fragments thereof, or nucleotide sequences that are substantially identical to SEQ. ID. NO.: 1 and SEQ. ID. NO.: 3 can be used in the immunogenic compositions described herein.

One embodiment of the invention provides an isolated and purified polynucleotide molecules encoding a VP6 protein, wherein the polynucleotide molecule is capable of hybridizing under moderate to stringent conditions to an oligonucleotide of 15 or more contiguous nucleotides of SEQ. ID. NO.: 1 and SEQ. ID. NO.: 3 including complementary strands thereto.

Construction of a synthetic gene encoding for a VP6 sequence is disclosed herein. In a preferred embodiment of the invention, the CJN VP6 nucleotide sequence is subjected to codon analyses, the codons that are not favorably used in E. coli cells are replaced by ones that are more abundant in E. coli genes generating a modified, codon-optimized, synthetic CJN VP6 gene sequence. Conversion of the synthetic gene sequence by computer translation programs confirm that the amino acid sequence remains unaltered. The process of codon optimization to enhance expression to overcome codon usage in host organisms is evident to those who are skilled in the art.

The nucleotide sequences of the disclosed invention have a myriad of applications. VP6 nucleotide sequences can be employed for the construction of recombinant cell lines, recombinant organisms, expression plasmids, and the like. Such recombinant nucleotide constructs can be used to express the recombinant rotavirus protein described herein. For example, the recombinant VP6 proteins can be expressed, purified, and used to prepare immunogenic subunit compositions. In another embodiment, recombinant VP6 proteins can be expressed in an organism, and the whole organism can be formulated into an immunogenic composition.

In one embodiment, an expression plasmid comprising a DNA molecule generated from the native nucleotide sequence encoding the CJN VP6 protein for expressing VP6 in bacterial cells is disclosed. In one embodiment, an expression plasmid comprising a DNA molecule generated from the codon-optimized nucleotide sequence encoding the CJN VP6 protein for expressing VP6 in bacterial cells is disclosed. In one embodiment, a DNA molecule including a VP6 gene, an adjuvant, and a purification tag is inserted into a suitable expression plasmid, which is in turn used to transfect or transform a suitable host cell. Preferably a DNA molecule including only the CJN VP6 gene is inserted into a suitable expression plasmid, which is in turn used to transfect or transform a suitable host cell. Representative expression plasmids include both plasmid and/or viral vector sequences. Suitable plasmids include pMAL/c2X, pMAL/P2 (New England Biolabs, Beverly Mass.), pET-Blue (Novagen, Madison Wis.), BLUESCRIPT (Stratagene, San Diego Calif.) plasmids, retroviral vectors, vaccinia viral vectors, CMV viral vectors, baculovirus vectors, and the like.

The CJN VP6 protein has been identified as having protective immunogenic properties (Example 26 ). The amino acid sequence of this protein is shown at SEQ. ID. NO.: 4. Sequences having variations from that of the CJN VP6 protein include those amino acid sequences resulting from minor genetic polymorphisms, differences between strains, those that contain natural amino acid substitutions, additions, and/or deletions, and from methods that introduce artificial amino acid substitutions, additions, and/or deletions known to those skilled in the art.

According to the present description, polynucleotide molecules encoding VP6 proteins encompass those molecules that encode a VP6 protein or peptide that shares identity with the sequence shown in SEQ. ID. NO.: 1 and SEQ. ID. NO.: 3. Such molecules preferably share greater than 30% identity at the amino acid level with the disclosed sequence. In preferred embodiments, the polynucleotide molecules can share greater identity at the amino acid level across highly conserved regions.

Amino acid sequences substantially the same as the sequences set forth in SEQ. ID. NO.: 2 and SEQ. ID. NO.: 4, are encompassed by the present description. A preferred embodiment includes polypeptides having substantially the same sequence of amino acids as the amino acid sequence set forth in SEQ. ID. NO.: 2 and SEQ. ID. NO.: 4, or functional fragments thereof, or amino acid sequences that are substantially identical to SEQ. ID. NO.: 2 and SEQ. ID. NO.: 4.

As would be evident to one skilled in the art, the polynucleotide molecules of the present disclosure can be expressed in a variety of prokaryotic and eukaryotic cells using regulatory sequences, plasmids, and methods well established in the literature.

VP6 proteins produced according to the present description can be purified using a number of established methods such as affinity chromatography using an anti-VP6 protein antibodies coupled to a solid support. Fusion proteins of an antigenic tag and a VP6 protein can be purified using antibodies to the tag. Optionally, additional purification is achieved using conventional purification means such as liquid chromatography, gradient centrifugation, and gel electrophoresis, among others. Methods of protein purification are known in the art and can be applied to the purification of recombinant VP6 proteins described herein.

Construction of VP6 fusion proteins is also disclosed. Fusion proteins will typically contain additions, substitutions, or replacements of one or more contiguous amino acids of the native VP6 protein with amino acid(s) from a suitable fusion protein partner. Such fusion proteins are obtained using recombinant DNA techniques well known to one skilled in the art, and are discussed more fully below. Briefly, DNA molecules encoding the hybrid VP6 proteins of interest are prepared using generally available methods such as PCR, and/or restriction digestion and/or ligation. The hybrid DNA is then inserted into expression plasmids and introduced into suitable host cells.

In one embodiment, the rotavirus VP6 fusion proteins contemplated by the present invention are composed of a suitable fusion protein partner in genetic association with a rotavirus protein or immunogenic fragment thereof. A fusion partner can be in fusion with the amino terminus and a different fusion partner with the carboxyl terminus of VP6. In another embodiment, the rotavirus VP6 fusion proteins are composed of a suitable fusion protein partner in genetic association with the amino terminus of VP6. In yet another embodiment, the rotavirus VP6 fusion proteins are composed of a suitable fusion protein partner in genetic association with the carboxyl terminus of VP6.

In one embodiment, a suitable fusion protein partner may actively prevent the assembly of the rotavirus fusion proteins into multimeric forms after the rotavirus fusion protein has been expressed. For example, the fusion protein partner should prevent the formation of dimers, trimers or virus-like structures that might spontaneously form if the rotavirus protein were recombinantly expressed in the absence of the fusion protein partner.

In another embodiment, a suitable fusion partner will facilitate the purification of the chimeric rotavirus fusion protein. A representative list of suitable fusion protein partners includes maltose binding protein, poly-histidine segments capable of binding metal ions, inteine, antigens to which antibodies bind, S-Tag, glutathione-S-transferase, thioredoxin, β-galactosidase, nonapeptide epitope tag from influenza hemagglutinin, a 11-amino acid epitope tag from vesicular stomatitis virus, a 12-amino acid epitope from the heavy chain of human Protein C, green fluorescent protein, cholera holotoxin or its A1, A2, A or B subunit, E. coli heat-labile holotoxin or its B subunit, CTA1-DD, streptavidin and dihydrofolate reductase.

In another embodiment, the fusion protein partner can be an adjuvant. The most potent adjuvants known to date are E. coli LT and CT. Both native and attenuated forms of LT and the closely related CT have been extensively assessed as mucosal vaccine adjuvants.

The invention is also directed toward producing rotavirus proteins for use in vaccines directed to protect immunized individuals from rotavirus infection and/or disease. Accordingly, the invention contemplates the use of an adjuvant, such as an immunogenic protein, effective to induce desirable immune responses from an immunized animal. Such a protein must possess those biochemical characteristics required to facilitate the induction of a protective immune response from immunized vertebrates while simultaneously avoiding toxic effects to the immunized animal.

In one embodiment of the present invention, rotavirus recombinant native or fusion proteins are mixed with an adjuvant such as a bacterial toxin. The bacterial toxin may be a cholera toxin. Alternatively, the rotavirus fusion protein may be mixed with the B subunit of cholera toxin (CTB). In another embodiment, an E. coli toxin may be mixed with the rotavirus fusion protein. For example, the rotavirus fusion protein may be mixed with E. coli heat-labile toxin (LT). The rotavirus fusion proteins of the present invention may be mixed with the B subunit of E. coli heat-labile toxin (LTB) to form a vaccine composition. Other adjuvants such as cholera toxin, labile toxin, tetanus toxin or toxoid, poly[di(carboxylatophenoxy)phosphazene] (PCPP), saponins Quil A, QS-7, and QS-21, RIBI (HAMILTON, Mont.), monophosphoryl lipid A, immunostimulating complexes (ISCOM), Syntax, Titer Max, M59, CpG, dsRNA, and CTA1-DD (the cholera toxin A1 subunit (CTA1) fused to a dimer of the Ig-binding D-region of Staphylococcus aureus protein A (DD)), are also contemplated.

As one embodiment, the protective immunity generated by the immunogenic compositions containing the recombinant rotavirus proteins of the present invention is a dominantly cell-mediated immune response. This immune response may interfere with the infectivity or activity of the rotavirus, or it may limit the spread or reproduction of the virus. The immune response resulting from vaccination with a vaccine containing the proteins of the present invention provides protection against subsequent challenge by a homologous or heterologous rotavirus.

The fusion partner or partners can be removed from the recombinant VP6 proteins for compliance with product safety. Proteolytic sites can be included for enzymatic cleavage and such procedures are well known to one skilled in the art.

Compositions

The immunogenic compositions described herein comprise a native recombinant rotavirus VP6 protein or, a codon-optimized rotavirus VP6 protein, immunogenic fragment(s) thereof, a rotavirus fusion protein, or immunogenic fragment(s) thereof, an adjuvant, and a pharmaceutically acceptable carrier. According to one embodiment of the present invention, a composition comprising a rotavirus protein or an immunogenic portion thereof is genetically associated with one or two fusion protein partners, and an adjuvant in a pharmaceutically acceptable carrier. This composition is administered to an individual (mammal) in whom an immune response directed against the rotavirus subunit protein is sought and protection against rotavirus infection and disease is desired.

The dosage regimen involved in a method for vaccination, including the timing, number and amounts of booster vaccines, will be determined considering various hosts and environmental factors, e.g., the age of the patient, time of administration and the geographical location and environment.

The rotavirus recombinant native or fusion proteins of the present invention may be used in a vaccine composition at a concentration effective to elicit an immune response from an immunized subject. The concentration of rotavirus proteins of the present invention may range from about 0.01 μg/ml to 1 mg/ml. In another embodiment, the concentration of rotavirus proteins used in a vaccine composition may range from about 0.1 μg/ml to 100 μg/ml. In yet another embodiment, the concentration of rotavirus proteins used in a vaccine composition may range from about 1.0 μg/ml to 10 μg/ml. In still another embodiment, the concentration of rotavirus proteins used in a vaccine composition may be about 8.8 μg/ml. These ranges are provided for the sake of guidance in practicing the present invention. It should be noted that other effective concentrations of recombinant rotavirus proteins may be determined by one skilled in the art using experimental techniques well known in the art.

The adjuvants described herein may be used in a vaccine composition at a concentration effective to assist in eliciting an immune response against the recombinant rotavirus fusion proteins of the present invention from an immunized subject. The concentration of adjuvant included in the vaccine compositions of the present invention may range from about 0.01 μg/ml to 1 mg/ml. In another embodiment, the concentration of adjuvant used in a vaccine composition may range from about 0.1 μg/ml to 100 μg/ml. In yet another embodiment, the concentration of adjuvant used in a vaccine composition may range from about 1.0 μg/ml to 100 μg/ml. In still another embodiment, the concentration of adjuvant used in a vaccine composition may be about 10.0 μg/ml. These ranges are provided for the sake of guidance in practicing the present invention. It should be noted that other effective concentrations of adjuvants may be determined by one skilled in the art using experimental techniques well known in the art.

The invention also encompasses immunization with a rotavirus fusion protein, a recombinant native protein, or a fragment or fusion fragment, and a suitable adjuvant contained in a pharmaceutically acceptable composition. Such a composition should be sterile, isotonic, and provide a non-destabilizing environment for the rotavirus fusion protein and the adjuvant. Examples of this are buffers, tissue culture media, various transport media and solutions containing proteins (such as BSA), sugars (sucrose) or polysaccharides.

The vaccine compositions of the present invention contain conventional pharmaceutical carriers. Suitable carriers are well known in the art. These vaccine compositions may be prepared in liquid unit dose forms. Other optional components, e.g., stabilizers, buffers, preservatives, excipients and the like, may be readily selected by one skilled in the art. However, the compositions may be lyophilized and reconstituted by the individual administering the vaccine prior to administration of the dose. Alternatively, the vaccine compositions may be prepared in any manner appropriate for the chosen mode of administration, e.g., intranasal administration, oral administration, etc. The preparation of a pharmaceutically acceptable vaccine, having due regard to pH, isotonicity, stability and the like, is within the skill of one skilled in the art.

One immunogenic composition of interest involves the formation of microparticles containing the immunogenic composition. The gastrointestinal tract provides a variety of morphological (e.g. epithelial cells, mucus) and physiological (e.g. enzymes, pH, bile salts) barriers to the soluble vaccine antigens. To overcome these barriers, microparticles have been designed to target a specialized cell population, e.g., the microfold (M) cells. M cells are follicle-associated epithelial cells that are specialized for transcytosis of microorganisms and particulates to the underlying organized mucosal lymphoid tissues. M cells contain a special lymphoid pocket into which transcytosed particles are released. These pockets are enriched with lymphocytes and are a part of the mucosal inductive sites. The effectiveness of oral vaccines has been shown to increase significantly by incorporation of vaccines into microparticles.

Routes of Administration

Also included in the present invention are methods of vaccinating humans against rotavirus infection and disease with the novel rotaviral proteins and vaccine compositions described herein. The vaccine compositions, comprising a full-length rotavirus protein, a rotavirus fusion protein, a recombinant native protein or fragments and fusion fragments, or mixtures of the above, and an adjuvant described herein may be administered by a variety of routes contemplated by the present invention. Such routes include intranasal, oral, rectal, vaginal, intramuscular, intradermal and subcutaneous administration, and needle-free routes of intramuscular, intradermal and subcutaneous administration.

Vaccine compositions for parenteral administration include sterile aqueous or non-aqueous solutions, suspensions or emulsions, the protein vaccine, and an adjuvant as described herein. The composition may be in the form of a liquid, a slurry, or a sterile solid which can be dissolved in a sterile injectable medium before use. The parenteral administration involving a syringe and needle or comparable means is preferably intramuscular. Needle free administration is preferably given as skin patches. The vaccine composition may contain a pharmaceutically acceptable carrier. Alternatively, the present vaccine compositions may be administered via a mucosal route, in a suitable dose, and in a liquid form. For oral administration, the vaccine composition can be administered in liquid, or solid form with a suitable carrier.

Doses of the vaccine compositions may be administered based on the relationship between the concentration of the rotavirus fusion protein contained in the vaccine composition and that concentration of fusion protein required to elicit an immune response from an immunized host. The calculation of appropriate doses to elicit a protective immune response using the rotavirus fusion protein vaccine compositions of the present invention are well known to those skilled in the art.

Methods of Administration

A variety of immunization methods are contemplated by the invention to maximize the efficacy of the rotavirus protein vaccine compositions described herein. In one embodiment, females of offspring-bearing age are immunized with the vaccines of the invention. In this embodiment, immunized females develop a protective immune response directed against rotavirus infection or disease and then passively pass this protection to an offspring by nursing. In another embodiment, newborns and infants are immunized with the vaccine compositions of the invention and shortly thereafter the nursing mother is immunized with the same vaccine. This two tiered approach to vaccination provides the newborn with immediate exposure to viral epitopes that may themselves be protecting. Nevertheless, the passive immunity supplied by the mother would augment the protection enjoyed by the offspring. This method would therefore provide the offspring with both active and passive protection against rotavirus infection of disease.

In still another embodiment, an individual is immunized with the vaccine composition of the invention subsequent to immunization with a multivalent vaccine. The immunization of a subject with two different vaccines may synergistically act to increase the protection an immunized individual would enjoy over that obtained with only one vaccine formulation. In this embodiment of the invention, the vaccine compositions serve as such a booster to increase the protection of the immunized individual against rotaviral infection or disease.

The following examples teach the generation of all types of rotavirus protein vaccine compositions. These examples are illustrative and are not intended to limit the scope of the present invention. One skilled in the relevant art would be able to use the teachings described in the following examples to practice the full scope of the present invention.

### EXAMPLES

### Example 1

Construction of a plasmid harboring the VP6 gene sequence of a mouse rotavirus strain.

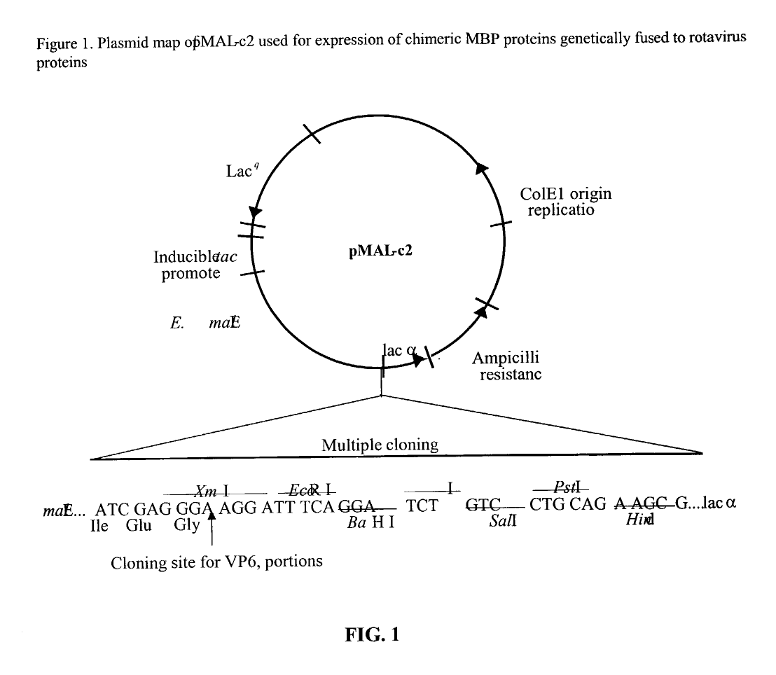

Recombinant plasmids pMAL-c2/EDIM6 were constructed using pMAL-c2 (New England Biolabs, Beverly Mass.) by insertion of cDNAs encoding full length VP6 of rotavirus strain EDIM (FIG. 1). The CDNA was synthesized by polymerase chain reaction (PCR) using the plasmid pGEM-3Z/EDIM6 as a template and gene specific primers determined by nucleotide sequencing of the gene inserts. The nucleotide sequences have been deposited into GenBank nucleotide sequence database and assigned with the Accession Numbers U65988.

The murine EDIM strain of rotavirus used for the construction of the pGEM recombinant plasmids was originally isolated from the stool of an infected mouse and adapted to grow in cell culture by passage in MA-104 cells in the laboratory. A triply plaque-purified isolate of the ninth passage was used to infect MA-104 cells to yield stock virus for RNA purification. To generate cDNAs of rotavirus genes encoding EDIM VP6, reverse transcription/polymerase chain reaction (RT/PCR) was carried out using purified genomic rotavirus RNA, a forward and a reverse primer obtained from the untranslatable regions of the gene. The cDNAs generated by RT/PCR were cloned into the Sma I site of the multiple cloning site of pGEM-3Z (Promega, Madison, Wis.). Ligation products were then transformed into E. coli strain BL2 1. White transformants carrying recombinant plasmids were selected by growing cells on LB agar plates containing IPTG (0.5 mM) and X-gal. Plasmids from individual colonies were purified and were analyzed by nucleotide sequencing.

In one embodiment, the unmodified, native cDNAs generated by PCR were inserted into the restriction site Xmn I of pMAL-c2, placing the inserted sequences downstream from and in genetic association with the E. coli malE gene, which encodes maltose binding protein (MBP), resulting in the expression of MBP fusion protein. In another embodiment, the VP6 cDNA was generated with additional six histidine codons just proximal to the stop codon. When the six histidine codons were included, the recombinant fusion MBP::VP6::His6 proteins have a hexahistidine (His6) fusion peptide at their carboxyl termini. The plasmid utilized the strong “tac” promoter and the malE translation initiation signals to give high-level expression of the fusion protein. pMAL-c2 contains the factor Xa cleavage site that is located downstream from the malE sequence to enable cleavage of the heterologous protein from MBP. The plasmid conveyed ampicillin resistance to recombinant bacteria and a lacZ-alpha gene sequence for blue-to-white selection of recombinants with inserts.

Following ligation of cDNA and XmnI-digested pMAL-c2, recombinant pMAL-c2 plasmids were transformed into E. coli strain BL21. White colonies of bacteria containing recombinant plasmids on an agar plate were then identified in the presence of IPTG and X-gal, and selected for further screening by PCR for gene identity and orientation. Nucleotide sequencing was used to ultimately confirm the authenticity of the rotavirus gene sequence.

### Example 2

Expression and purification of chimeric EDIM VP6 proteins.

Expression of chimeric EDIM VP6 in E. coli cells have been previously described. Choi et al., J Virol, 73:7574-7581, (1999). Choi et al., J Virol, 74:11574-11580, (1999). Recombinant bacteria were grown as an overnight culture (37° C., shaken at 215 rpm) in rich broth (tryptone, 10 gm; yeast extract, 5 NaCl, 5 gm; glucose, 2 gm and 100 mg of ampicillin per liter). On the following day, 10 ml of overnight cell culture were inoculated into 1 liter of rich broth containing glucose and ampicillin. The culture was grown until the optical density A₆₀₀ reached 0.6. IPTG was then added to 0.3 mM______to induce expression of fusion protein. Growth was continued for 3 hours.

Cells were harvested by centrifugation (4,000 g; 20 min. at 4° C.), resuspended in PBS, and subjected to centrifugation. The pellet was frozen at −20° C., thawed slowly in cold water, and resuspended in a total of 50 ml of buffer L (5 mM NaH₂PO₄, 10 mM Na₂HPO₄, 30 mM NaCl, 10 mM 2-beta mercaptoethanol and 0.2% Tween 20, 1 mM PMSF, 25 mM benzamidine, and 200 mg/L of lysozyme). After digestion for 15 min. at room temperature (rt), the suspension was sonicated for three 30 second bursts (BioSonic IV, 50% power setting) while placed in an ice/water bath. NaCl (26.5 mg/ml) and RNase A (5 μl of 10 mg/ml) were added to each 10 ml of sonicate which was then centrifuged (54,000 g, 30 min.) to obtain a supernatant containing a crude preparation of fusion protein.

Fusion proteins in the crude preparation were purified by affinity chromatography. Amylose resin (New England Biolab, Beverly Mass.) and Talon resin when the His6 tag was present, was prepared by placing 25 ml of the packed resin in a 250 ml centrifuge tube and washed twice with eight volumes of buffer C (Buffer L containing 0.5 M NaCl). For each wash, the mixture was rocked for 30 min. at 4° C., and the resin was recovered by centrifugation (2,100 g, 5 min.). The supernatants, which contained the fusion proteins, were mixed with amylose resin for 2 hours in a 500 ml flask on a magnetic stirrer. After centrifugation (2,100 g, 5 min.), the resin was recovered, then resuspended in 50 ml of buffer C, rocked for 30 min. and finally centrifuged to recover the resin. The resin was washed in this manner for a total of 3 times and finally washed overnight with 500 ml of buffer C.

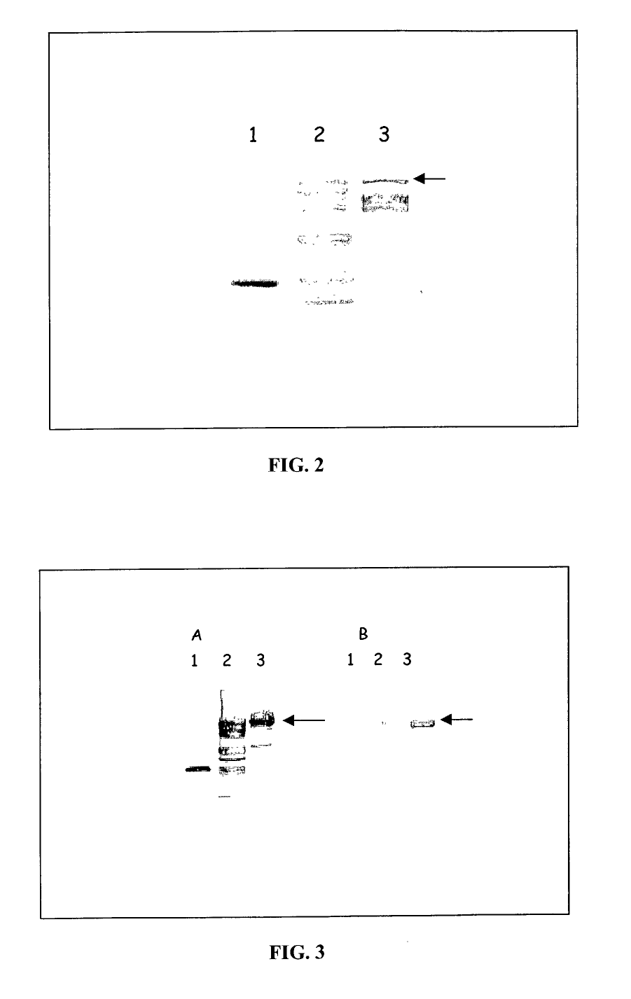

On the following day, the resin was recovered by centrifugation (2,100 g, 5 min.) and resuspended in 50 ml of buffer D (50 mM Tris-HCl, pH 7.5; 50 mM NaCl; 1 mM EDTA; 10 mM 2-beta mercaptoethanol; 1 mM PMSF), and rocked for 30 min. The resin was spun down and the bound fusion proteins were eluted from the resin with 250 ml of 15 mM maltose in buffer D for 2 hours. The resin was recovered by centrifugation (2,100 g, 5 min.) and the supernatant containing the fusion proteins was subjected to buffer exchange to PBS and was simultaneously concentrated by ultrafiltration using a stirred-cell concentrator (Amicon, Beverly Mass.; model 8400). When the protein was further purified using Talon resin, urea was added to the protein solution to a final concentration of 8M and the pH adjusted to pH 7. The protein was purified according to the instructions of the resin manufacturer (BD Biosciences/Clontech, Palo Alto Calif.). The purified fusion proteins were analyzed by immunoblot analyses (FIG. 2).

### Example 3

Biochemical characterization of MBP::VP6 fusion protein

It has been shown that recombinant VP6 expressed by the baculovirus expression system forms structures that resemble double-layered rotavirus particles when examined by electron microscopy. Purified MBP::VP6 fusion protein was analyzed by sucrose gradients to determine if these fusion proteins assembled into organized structures resembling virus particles that could be fractionated in a sucrose gradient. MBP::VP6 was subjected to centrifugation (SW 50, 35,000 g, 60 min.) through a 4 ml sucrose gradient (20-50%) on a 1 ml cesium chloride cushion (60%). A total of 16, 300-μl fractions were collected. Distribution of MBP::VP6 in the sucrose gradient and cesium chloride cushion was analyzed by immunoblot analysis and distribution of virus particles was analyzed by silver nitrate staining of the SDS-gel (FIG. 3). The results showed that MBP::VP6 remained in the top 4 fractions of the gradient, while double-layered virus particles devoid of VP4 and VP7 were recovered from fraction #11 to #12 of the sucrose gradient and in the cesium chloride cushion (fraction #16). The difference in the distribution behavior of MBP::VP6 in the gradient indicated that the fusion protein does not form virus-like structures.

### Example 4

Method of Vaccination and Challenge.

Six-week-old virus antibody free female BALB/c mice were purchased from Harlan Sprague-Dawley (Indianapolis, Ind.). Animals were housed four animals to a cage in sterile micro barrier cages. Four to ten animals were included in each group. Animals were ear tagged and a blood and stool specimen was collected from each animal prior to vaccination.

Expressed fusion protein of EDIM VP6 s used as the immunizing antigens. Protein concentration was calculated to be 176 ng/μl. Animals received 50 μl of VP6 (8.8 μg) per dose. Animals received either one immunization, or two, or three doses separated by two-week intervals.

The adjuvant used was E. coli LT (R192G) at 1 mg/ml received from Dr. John Clements (Tulane University). The LT was resuspended in deionized H₂O and 10 mM CaCl₂. Intranasal inoculations included 10 μg LT with antigen. Adjuvant and antigens were mixed prior to immunization. Animals were immunized intranasally (i.n.) by lightly anesthetizing with metofane and instilling approximately 5 μl per nostril until the entire dose was delivered.

### Example 5

Collection of specimens for analysis of the vaccine efficacies and immune response from subjects immunized with edim vp6 fusion proteins

Regardless of the number of doses, formulation or route used, the immunogenicity and efficacy were evaluated using the protocol described as follows. Four weeks after the last immunization, animals were bled and a stool specimen was collected from each animal to measure antibody responses. Animals were challenged with 100 μl of a 1:25 dilution of EDIM P9 12/15/97 1×10⁷ ffu/ml to give a dose of 4×10⁴ ffu or 100 ID₅₀. Stool specimens (two pellets in 0.5 ml of Earl's Balanced Salt Solution (EBSS)) were collected from each mouse for seven days and stored at −20° C. Rotavirus antigen was measured in the stools by EIA to determine shedding. Twenty-one days after challenge, sera and stool specimens were obtained again to measure antibody responses.

### Example 6

EIA Method to Measure Rotavirus Antigen in Stool to Determine Shedding.

Stool specimens collected from mice immunized in experiments in Example 4 were thawed, homogenized and centrifuged (500 g, 10 min.). For rotavirus antigen determination, 96-well EIA plates (Corning Costar Co., Corning, N.Y.) were coated overnight at 4° C. with 100 μl per well of either rabbit antibody to rotavirus (duplicate positive wells) or preimmune rabbit serum (duplicate negative wells). Plates were washed and 50 μl of stool supernatant was added to duplicate wells coated with each antibody. After one hour incubation at 37° C. on a rotation platform, the plates were washed and 50 μl normal goat serum (Vector Laboratory, Inc., Burlingame, Calif.) diluted 1:100 in phosphate-buffered saline containing 5% nonfat dry milk (PBS-M) was added for 15 minutes at room temperature. Fifty microliters of guinea pig antibody to rotavirus diluted 1:500 in PBS-M containing a 1:50 dilution of normal rabbit serum (DAKO, Carpinteria, Calif.) was added and incubated for 30 minutes. The plates were washed and 50 μl of a 1:200 dilution of biotinylated goat anti-guinea pig IgG (Vector) in PBS-M containing a 1:50 dilution of normal rabbit serum was added and incubated 30 minutes. After washing plates, 50 μl of a 1:100 dilution of peroxidase-conjugated avidin-biotin (Vector) in wash buffer was added and incubated 30 minutes. The plates were washed and 50 μl substrate (o-phenylenediamine with H₂O₂ in citric acid-phosphate buffer) was added and incubated at room temperature for 15 minutes. The reaction was stopped with 75 μl of 1.0 M H₂SO₄. The absorbance at 490 nm was measured and the net optical densities were determined by subtracting the average of the negative wells from the average of the positive wells. The specimen was considered positive for rotavirus if the average absorbance of the positive wells was greater than or, equal to two times that of the negative wells and greater than or equal to 0.15.

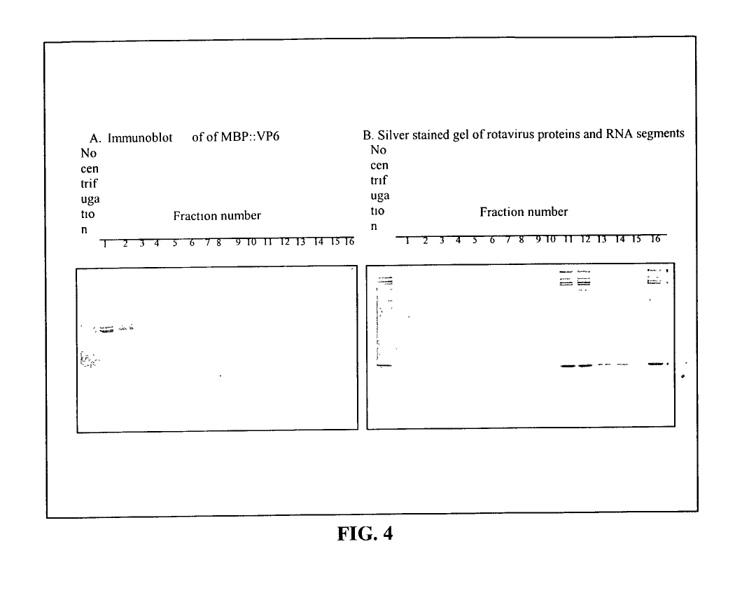

A time course of fecal shedding of rotavirus in mice challenged with EDIM is shown in FIG. 4. As can be seen from the figure, the incidence of fecal shedding increased from the first day after EDIM challenge in the control mice until reaching a maximum value on the fourth day after challenge. In contrast, mice vaccinated with the VP6 rotavirus fusion protein produce little fecal shedding over the same period of time. These data clearly show that intranasal vaccination of mice with rotavirus fusion vaccine composition greatly reduced the incidence of fecal shedding of virus after rotavirus EDIM challenge.

### Example 7

EIA Method to Measure Serum Rotavirus IgG and IgA and Stool Rotavirus IgA.

Serum rotavirus IgA and IgG and rotavirus stool IgA were measured as follows. EIA plates (Corning Costar Co., Corning, N.Y.) were coated overnight at 4° C. with anti-rotavirus rabbit IgG. After washing with phosphate buffered saline plus 0.05% Tween 20, 50 μl of EDIM viral lysate or mock-infected cell lysate were each added to duplicate positive and duplicate negative wells and plates were incubated for one hour at 37° C. on a rotation platform. After washing the plates, 50 μl of serial two-fold dilutions of pooled sera from EDIM infected mice containing concentrations of 160,000 or 10,000 units/ml of rotavirus IgG or IgA, respectively, were added to duplicate wells coated with either EDIM-infected or uninfected MA-104 cell lysates to generate a standard curve. Serial 10-fold dilutions of mouse sera to be tested were also added to duplicate wells of each lysate and incubated for 1 hour. This was followed by sequential addition of biotin-conjugated goat anti-mouse IgG or IgA (Sigma Chemical Co., St. Louis, Mo.), peroxidase-conjugated avidin-biotin (Vector Laboratories), and o-phenylenediamine substrate (Sigma Chemical Colo.). Color development was stopped after fifteen minutes with 1M H₂SO₄ and the A₄₉₀ was measured. Titers of rotavirus IgG or IgA, expressed as units/ml, were determined from the standard curve generated by subtraction of the average A₄₉₀ values of the duplicate cell lysate wells from the average of the wells coated with EDIM lysate.

For determination of stool rotavirus IgA, two stool pellets were collected into 0.5 ml of EBSS, homogenized, and centrifuged (1,500 g, 5 min.). Stool rotavirus IgA was then measured by the method described above.

Table 1 shows that mice immunized with the recombinant MBP::VP6 rotavirus fusion protein vaccines generated an immune response directed against the VP6 fusion protein. Both serum IgG and IgA responses were noted. The serum IgG responses were higher than those of the IgA responses. In control mice which were immunized with the adjuvant alone, no VP6-specific antibodies were detected.

TABLE 1
Geometric mean titers of rotavirus antibodies following immunization of
BALB/c mice by two intranasal immunizations with the vaccine
composition containing MBP::EDIM-VP6 and LT(192G).

| | |
|---|---|
| VP-6 Specific | Vaccine composition |

| Antibody (U/ml) | LT(R192G) | MBP::VP6 and LT(192G) |
|---|---|---|
| Serum IgG | 0 | 200,566 |
| Serum IgA | 0 | 954 |
| Stool IgA | 0 | 10 |

### Example 8

Immunoblot analysis to show that intranasal immunization with the vaccine composed of MBP::EDIM-VP6 induced VP6-specific antibodies.

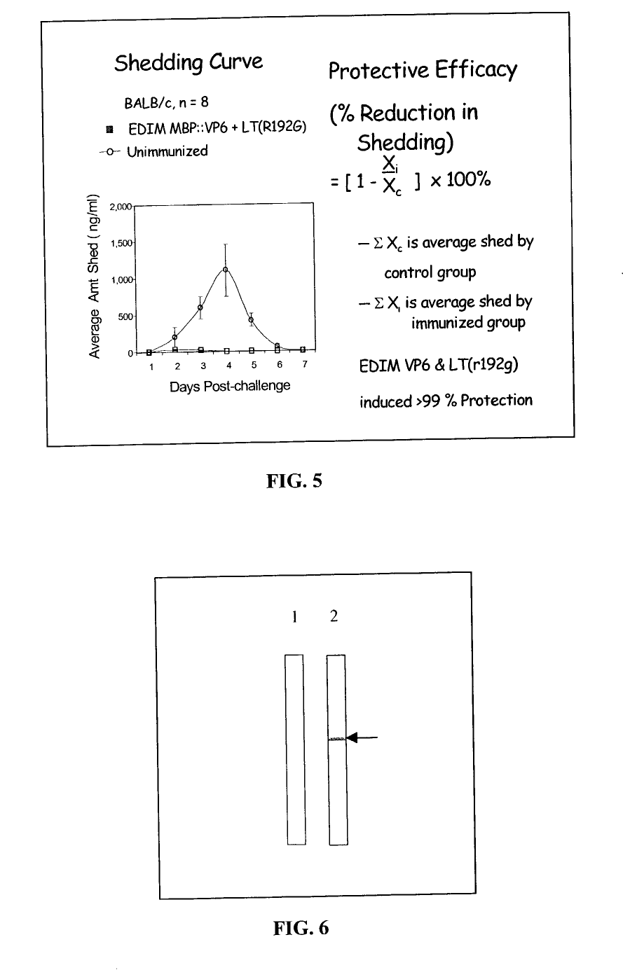

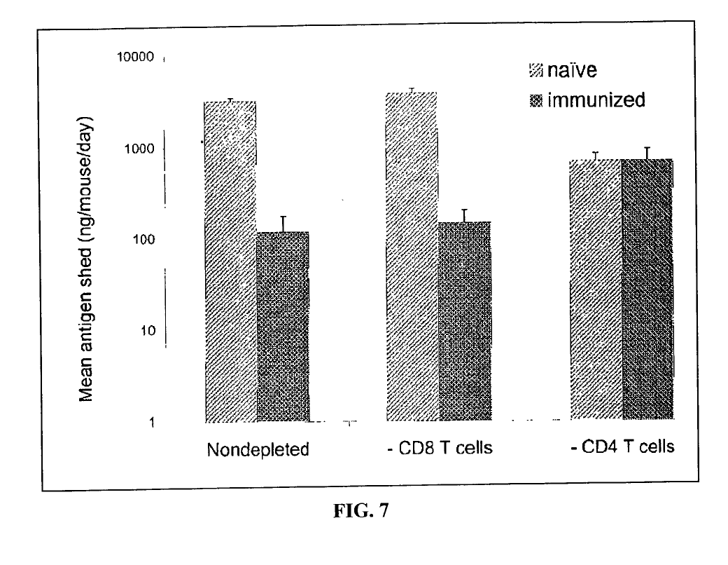

Serum samples from mice immunized with vaccines were analyzed for rotavirus VP6-protein-specific antibodies by immunoblot analyses. Cesium chloride gradient-purified rotavirus particles were subjected to SDS-polyacrylamide gel electrophoresis. Separated rotavirus proteins were blotted to a nitrocellulose sheet and cut into strips each of which contained 3 μg of rotavirus proteins. The strips were blocked with 5% skim milk in Tris-HCl buffer (TBS, 50 mM Tris-HCl, pH 7.5, 0.9% NaCl). The strips were then incubated with antisera obtained from immunized mice. After washing with 0.1% Tween-20 in TBS, the strips were incubated with goat anti-mouse IgG conjugated to alkaline phosphatase (Life Technologies, Gaithersburg, Md.). The strips were washed with TBS and then incubated with 4-chloro-3-indolylphosphate and nitroblue tetrazolium (Life Technologies, Gaithersburg, Md.) to visualize bound antibodies (FIG. 5).

### Example 9

One immunization is sufficient to elicit protective immunity.

To determine the minimum dose that could provide the same level of protection as higher number of immunizations, mice were immunized intranasally with 1, 2 or 3 doses of MBP::VP6 (8.8 μg/dose) using LT as adjuvant (Table 2). For the latter two groups, doses were given 14 days apart. Measurement of serum rotavirus-specific IgG indicated that the levels of IgG induced by three doses (GMT=417,604 U/ml) was higher than two doses (GMT=122,839 U/ml), which in turn was higher than one dose (GMT=32,843 U/ml; Table 4). Serum IgA titers for 3 doses were higher (GMA=1,185 U/ml) than 2 doses (GMT=256 U/ml) or 1 dose (GMT=243 U/ml). Larger titers of stool IgA could be detected in mice receiving 3 doses (GMT=77 U/ml) than 2 doses (GMT=24 U/ml). Only a few animals receiving 1 dose developed measurable stool rotavirus IgA (GMT-12 U/ml).

TABLE 2
One, two or three doses of intranasal vaccine containing MBP::EDIM-VP6
and LT(R192G) induced VP6-specific antibodies in immunized mice.

| VP6-specific | Number of doses | | |
|---|---|---|---|
| antibodies (U/ml) | 1 | 2 | 3 |
| Serum IgG | 32,843 | 122,839 | 417,604 |
| Serum IgA | 243 | 256 | 1,185 |
| Stool IgA | 12 | 24 | 77 |

Although the immunological responses differed between the 1, 2 and 3 dose protocols, animals were shown to be protected by a single vaccination. Analyses of the quantities of rotavirus antigen shed following rotavirus challenge one month after the last or only immunization indicated that 1, 2 or 3 doses of the vaccine resulted in almost 100, 98 and 98% reduction in shedding, respectively (Table 3). Therefore, one dose of MBP::VP6 was sufficient to induce essentially complete protection and protection appeared to be independent of the titer of specific antibodies.

TABLE 3
One immunization of an intranasal vaccine composition containing
MBP::EDIM-VP6 and LT(R192G) is sufficient to induce protection.

| Number of | % Reduction in shedding of rotavirus antigens |
|---|---|
| immunizations | in stools of immumunized mice |
| 1 | >99 |
| 2 | 98 |
| 3 | 98 |

### Example 10

Protection induced by the composition containing MBP::EDJM-VP6::His6 did not wane for at least 6 months.

In the typical immunization protocol, mice were challenged 1 month after the last immunization. For this study, the time between the last immunization and challenge was extended to 3 months to determine whether the degree of protection (quantity of virus shed after challenge) is reduced with time. Mice given two intranasal immunizations with MBP::VP6 (8.8 μg/dose) and LT(R129G) separated by a 2 week interval were found to be equally protected at 3 months (99.7%) or 1 month (97.8%) after the immunization (Table 4). This finding demonstrates that protection is not rapidly lost after immunization, an important finding regarding the utility of VP6 as a vaccine candidate.

TABLE 4
Protection induced by the composition containing MBP::EDIM-VP6::His6
did not wane for at least 6 months.

| Time of virus challenge | % Reduction in |
|---|---|
| after the last | shedding of rotavirus |
| immunization (months) | antigens in stools of immunized mice |
| 1 | 99.7 |
| 6 | 99.8 |

### Example 11

Induction of protective immunity by another mucosal route.

To determine whether MBP::VP6 is protective if delivered by a mucosal route other than intranasally, groups of mice were immunized orally with 2 inoculations of MBP::VP6 (8.8 μg per inoculation), either with or without LT(R192G). Another group was immunized intranasally with this fusion protein and LT(R192G) for comparison. Immunized mice were challenged with murine rotavirus 1 month after the last immunization and the percent reduction in viral shedding was calculated (Table 5). Oral immunization with MBP::VP6 and LT(R192G) induced good protection (85% reduction in shedding) but this reduction was significantly (P<0.001) less than after intranasal immunization (99%). Therefore, intranasal was more effective than oral immunization; however, it is possible that the two routes may be used concomitantly to increase protection, a possibility to be examined in future experimentation. Induction of protection by oral inoculation, as in the case of intranasal immunization, was dependent on the presence of LT(R192G), which reemphasized the requirement for an adjuvant to be used in conjunction with the VP6 vaccine.

TABLE 5
Oral immunization of EDIM-VP6/LT(R192G) induced good protection.

| | % Reduction in |
|---|---|
| | shedding of rotavirus antigens in stools |
| Route of immunization | of immunized mice |
| Oral | 94 |
| Intranasal | 99 |

### Example 12

Effect of different adjuvants on protection.

The adjuvant used in the vaccine formulation had been primarily LT(R192G) owing to its powerful adjuvanticity in eliciting protection in immunized animals against rotavirus antigen shedding following oral rotavirus challenge. However, the promiscuous binding of bacterial enterotoxins via their B subunits to cells at mucosal sites may prevent their use in humans. In view of safety concerns, safer adjuvants for intranasal immunization were sought and these adjuvants were evaluated orally.

Representatives of four of five types of adjuvants (Table 6) that are distinguished by their mechanisms of action, physical and chemical properties were evaluated. The adjuvants were Adjumer®, CpG ODN (oligodeoxynucleotides), CTA1-DD, LT(R192G), and QS-21®. Adjumer® (Parallel Solutions, Inc.) is a water-soluble, synthetic polyphosphazene polymer. CpG ODN (Coley Pharmaceuticals, Wellesley Pa.) is a nuclease-resistant synthetic oligonucleotide that contains a 6 base-pair motif consisting of the unmethylated, immunostimulatory CpG dinucleotide motif. CTA1-DD (supplied by Dr. Niles Lycke, University of Goteborg) is a chimeric protein containing the A1 subunit of cholera toxin genetically fused to two copies of the immunoglobulin-binding fragment of protein A derived from S. aureus. QS-21® is a water soluble saponin purified from the bark of the South American Quillaja saponaria Molina tree (Wyeth/Lederle). LT(R192G) (supplied by Dr. John Clements, Tulane University) is an attenuated form of the E. coli heat-labile toxin. Adjumer®, CpG-containing oligonucleotides and QS21 are potent vaccine adjuvants and have been shown to be safe in human clinical trials, and CTA1-DD have been found safe in preclinial studies.

TABLE 6
Effect of different adjuvants co-administered with MBP::EDIM-VP6
delivered intranasally or orally on protection against shedding of rotavirus
antigens in BALB/c mice.

% Protection from
rotavirus shedding

| Adjuvant | Intranasal | oral |
|---|---|---|
| Unimmunized | — | — |
| No adjuvant | 16 | 0 |
| LT(R192G) | >99 | 94 |
| CTA1-DD | 95 | 35 |
| Adjumer | 80 | 28 |
| QS-21 | 43 | 71 |
| CpG ODN | 74 | 55 |

Groups of 8-16 BALB/c mice were immunized either intranasally or perorally with two doses, separated by a two-week interval, with vaccines containing EDIM VP6 (9 μg) and one of the adjuvants (10 μg LT(R192G), 10 μg CTA1-DD, 50 μg Adjumer®, 20 μg QS-21® or 10 of CpG ODN) (Table 6). The amounts of adjuvants used were based on those determined for other antigens by the suppliers. The adjuvants and MBP::EDIM-VP6 were mixed just prior to inoculation. Immunized mice were challenged 4 weeks after the second immunization and protection from shedding was determined for each group. The results showed that LT(R192G) is the only powerful adjuvant for both intranasal and oral delivery of VP6 proteins. Following intranasal immunization, LT(R192G), CTA1-DD, Adjumer® and CpG ODN were found to induce significant (P<0.05, ANOVA and Tukey's multiple group test) protection against oral EDIM rotavirus challenge. However, only LT(R192G) and CTA1-DD had significant effects on VP6-induced reduction in fecal rotavirus shedding. Oral immunization with these adjuvants was less effective. Only LT(R192G) and QS-21® reduced shedding of rotavirus antigen when compared to the unimmunized group after peroral immunization, and only the group immunized with LT(R192G) shed significantly less (P<0.05) than the group that received VP6 alone.

When delivered by either mucosal route, while CTA1-DD stimulated significant protection only by the intranasal route. An explanation for the difference in efficacies between these two adjuvants (i.e., LT(R192G) and CTA1-DD) is that bacterial holotoxins are extremely stable in acidic environments and resistant to proteolysis in the gastrointestinal tract. Resistance to acid pH (between pH 2 and 3.9) and trypsin (1 mg/ml) is attributed to the stable pentameric B subunits of holotoxins.

The adjuvants Adjumer and CpG ODNs are chemically synthesized. Because new candidates of these two types of adjuvant are constantly being designed and evaluated, they are the most promising for replacing LT (R192G) in the vaccine compositions of the present invention.

### Example 13

Effect of dosage on protection.

The effect of dosage (1.76 μg and 8.8 μg) on protection by intranasal immunization with MBP::VP6 and LT (R192G) was examined (Table 7). Although mice immunized with two 1.76 μg dosages plus LT (R192G) of chimeric VP6 appeared to be nearly as well protected as those administered two 8.8 μg-doses, (protective levels were 94 and 99%, respectively) the 94% protection level was significantly (P=0.0003) lower than the 99% protection level.

An even smaller dosage (CJN VP6. 0.09 μg, Example 26) may be reached which will still induce maximum protection by the composition containing chimeric and LT(R192G).

TABLE 7
The dosage can be reduced from 8.8 ug to1.76 ug without affecting the
efficacy of protection induced by the formulation containing MBP::EDIM-
VP6 and LT(R192G)

| Dosage of | |
|---|---|
| MBP::VP6 (ug) | % Protection from rotavirus shedding |
| 8.8 | 99 |
| 1.76 | 94 |

### Example 14

Intranasal formulation containing full-length MBP::EDIM-VP6 induced almost complete protection in three inbred strain of mice.

Because of genetic diversity in the haplotypes of human populations, experiments were performed to determine the level of protection that could be stimulated by the mouse rotavirus-derived MBP::EDIM-VP6 in inbred mice having different haplotypes. In addition to the H-2^{d}-haplotype BALB/c mice, the mouse strains 129, DBA/2, and C3H, which have the haplotypes H-2^{b}, H-2^{d}, and H-2^{k}, respectively, were used (Table 8). These mice were susceptible to infection by EDIM rotavirus. After these inbred mice were immunized intranasally with 2 doses of MBP::EDIM-VP6 (9 μg) and LT (R192G) (10 μg]), all 3 strains of mice were found to develop >99% protection from rotavirus shedding. Intranasal immunization with MBP::EDIM-VP6 and LT(R192G) of inbred strains of mice having different haplotypes developed >99% protection from rotavirus shedding.

TABLE 8
Protection of Inbred mouse strains following intranasal immunization
with 9 ug doses of EDIM VP6 and 10 ug of LT(R192G).

| | | % Protection from shedding |
|---|---|---|
| Mouse strain | Haplotype | of rotavirus antigen |
| BALB/c | H-2^{d} | 98.7 |
| DAB/2 | H-2^{d} | 99.9 |
| 129 | H-2^{b} | 99.7 |
| C3H | H-2^{k} | 99.8 |

### Example 15

Serum rotavirus IgG responses in inbred mouse strains following intranasal immunization with the present invention.

As markers of immunogenicity, the VP6-specific-serum IgG antibodies in mice immunized in Example 13 were titered by ELISA. All inbred strains of mice developed high titers of specific response following intranasal immunization with the present invention containing the mouse EDIM VP6 (Table 9).

TABLE 9
Inbred strains of mice developed VP6-specific IgG titers following
intranasal immunization with the MBP::EDIM-VP6 and LT(R192G)
composition.

| | | | IgG titers |
|---|---|---|---|
| Mouse Strain | Haplotype | N | (ngs/ml) |
| BALB/c | H-2^{d} | 8 | 31,374 |
| DBA/2 | H-2^{d} | 5 | 436,541 |
| C57BL/6 | H-2^{b} | 8 | 60,416 |
| 129 | H-2^{b} | 6 | 139,838 |
| C3H | H-2^{k} | 6 | 42,810 |

N = Number of mice in each group.

### Example 16

Protection against EDIM shedding does not require B cell function.

B-cell deficient VTMT mice were vaccinated intranasally with two doses (8.8 μg/dose) of MBP::VP6 with LT. As expected, no rotavirus IgG, IgA or IgM was detected in the sera of any of these mice during this study. Analyses of virus shedding indicated that the subunit vaccine was as protective in these mice as was found with immunologically normal BALB/c mice (Table 10). This finding suggested that the vaccine could induce protection by a mechanism that did not require rotavirus antibodies. The mechanism is therefore not antibody dependent.

TABLE 10
Protection induced by the intranasal formulation containing MBP::VP6
and LT(R192G) does not require B cell functions

| Mouse strain | % Protection from rotavirus shedding |
|---|---|
| BALB/c | 97.0 |
| μMt (B cell deficient) | 99.7 |

### Example 17

CD8 T cells are not required for MBP::VP6-mediated immunity.

Studies using rotavirus particles for intranasal immunization have shown that CD8 cells are not needed for protection. Experiments were performed to determine whether immunization with VP6 can also mediate CD8-independent protection (FIG. 6). Effects of either CD8 T-cell depletion on shedding of rotavirus antigen in either naive or VP6-immunized, B-cell-deficient J_{H}D mice during the 7 days after EDIM challenge. Groups of six J_{H}D mice were either not immunized or i.n. immunized with two doses of MBP::VP6 and LT(R192G) separated by 2 weeks. Starting at 24 days after the second dose, some groups of mice were depleted of either CD8 T cells by daily (4 consecutive days) injections with MAbs specific for each cell type. On day 28 after the second dose, all mice were challenged with 1,000 SD₅₀ of wild-type EDIM and monitored daily for shedding of rotavirus antigen during the following 7 days. Two additional MAb injections were administered during the 7-day analysis period. The results are shown in FIG. 5 and represent the average amounts in nanograms (ng) of rotavirus antigen shed/mouse/day during the 7-day period, with standard deviations shown by the error bars.

The experiment clearly shows that depletion of CD8 cells in immunized mice has no effect on protection induced by the formulation containing EDIM VP6 protein and LT(R192G).

### Example 18

CD4 cells are most important for VP6-mediated protection.

Based on results found with μMt mice (Example 16), protection stimulated by VP6 was found not to be dependent on antibody production. Furthermore, protection following immunization with MBP::VP6 was not dependent on CD8 cells. This would leave CD4 cells as the most likely memory cells involved in protection. The effects of CD4 T-cell depletion on shedding of rotavirus antigen in either naive or VP6-immunized, B-cell-deficient J_{H}D mice during the 7 days after EDIM challenge were studied (FIG. 6). Groups of six J_{H}D mice were either not immunized or i.n. immunized with two doses of MBP::VP6 and LT(R192G) separated by 2 weeks. Starting at 24 days after the second dose, some groups of mice were depleted of either CD8 or CD4 T cells by daily (4 consecutive days) injections with MAbs specific for each cell type. On day 28 after the second dose, all mice were challenged with 1,000 SD₅₀ of wild-type EDIM and monitored daily for shedding of rotavirus antigen during the following 7 days. Two additional MAb injections were administered during the 7-day analysis period. The results represent the average amounts in nanograms (ng) of rotavirus antigen shed/mouse/day during the 7-day period, with standard deviations shown by the error bars. The results clearly showed that CD4-T cell depletion abrogates protection induced by the present invention that contains recombinant VP6 and LT(R192G).

### Example 19

Construction, expression and purification of truncated chimeric vp6 fusion protein fragments.

This example illustrates that chimeric VP6 fragments may be produced by the method described in Examples 1 and 2 using genetic engineering techniques. To produce a minimal subunit vaccine while retaining the original protective efficacy, three plasmids, pMAL-c2/EDIM6_{AB}, pMAL-c2/EDIM6_{BC} and pMAL-c2/EDIM6_{CD}, were constructed to express truncated forms of VP6, wherein the truncated forms of VP6 contain immunogenic fragments of a rotavirus protein. Recombinant plasmids pMAL-c2/EDIM6_{AB}, PMAL-c2/EDIM6_{BV} and pMAL-c2/EDIM6_{CD}, containing truncated forms of VP6 were constructed using the same strategy that was used for the construction of pMAL-c2/EDIM6, as seen in Example 1. These plasmids expressed MBP::VP6_{AB} containing amino acids 1 to 196, MBP::VP6_{BC} containing amino acid 97 to 297 and MBP::VP6_{CD} containing amino acids 197 to 397.

To construct these plasmids, cDNAs were synthesized by polymerase chain reaction (PCR) using pMAL-c2/EDIM6 (see Example 1) as the template. The gene specific primers used for construction and the regions of VP6 cloned are summarized in Table 11.

Once constructed, the plasmids encoding the truncated VP6 fragments were introduced into bacteria for protein expression. Recombinant bacteria containing pMAL-c2/EDIM6_{AB}, PMAL-c2/EDIM6_{BC} and PMAL-c2/EDIM6_{CD} were grown and recombinant proteins were expressed and purified as described above (Example 2).

TABLE 11

rimers used to clone pMAL-c2/MBP_{AB}, pMAL-c2/
EDIM6_{BC} and pMAL-c2/EDIM6_{CD}

| | Name of Fusion | |
|---|---|---|
| Plasmid | Protein | Primers |
| pMAL-c2/MBP_{AB} | MBP::VP6_{AB} | Forward primer: ATG |
| | | GAT GTG CTG TAC TCT |
| | | ATC SEQ. ID. NO.6 |
| | | Reverse primer: TCA |
| | | CGA GTA GTC GAA TCC |
| | | TGC AAC SEQ. ID. NO.7 |
| pMAL-c2/EDIM6_{BC} | MBP::VP6_{BC} | Forward primer: ATG |
| | | GAT GAA ATG ATG CGA |
| | | GAG TCA SEQ. ID. NO.8 |
| | | Reverse primer: TCA |
| | | GAA TGG CGG TCT CAT |
| | | CAA TTG SEQ. ID. NO.9 |
| pMAL-c2/EDIM6_{CD} | MBP::VP6_{CD} | Forward primer ATG |
| | | TGC GCA ATT AAT GCT |
| | | CCA GCT SEQ. ID. NO.10 |
| | | Reverse primer: TCA |
| | | CTT TAC CAG CAT GCT |
| | | TCT AAT SEQ. ID. NO.11 |

### Example 20

Vaccination and challenge of mice using truncated fragments of VP6 fusion proteins.

Six-week-old immunologically naive female BALB/c mice (Harlan Sprague) were used to study the ability of the various VP6 fragments to elicit a protective response from vaccinated mice. Blood and stool specimens were collected from the animals prior to vaccination. Animals were immunized intranasally with 8.8 μg of fusion protein vaccines (MBP::VP6, MBP::VP6_{AB}, MBP::VP6_{BC} or MBP::VP6_{CD}) in a 50-μl volume. Animals, which received three doses, were immunized at biweekly intervals. The animals received 10 μg of the adjuvant LT(R192G) with the vaccines.

Four weeks after the last immunization, the animals were bled and stool specimens were collected to measure antibody responses. Each animal was challenged with a 100 ID₅₀ s dose, which is equivalent to 4×10⁴ ffu, of EDIM virus (Lot number: P9 12/15/97). Two stool pellets were collected in 0.5 ml of Earl's Balanced Salt Solution (EBSS) from each mouse for seven days and stored at −20° C. Rotavirus antigen was measured in the stools by EIA to determine shedding. Twenty one days after challenge, sera and stool specimens were obtained again to measure antibody responses. The results in this example are summarized in Table 12 showing that the chimeric VP6 fragments, MBP::VP6_{AB}, MBP::VP6_{BC} MBP::VP6_{CD} induced 80, 92 and >99% protection, respectively, in mice.

TABLE 12
Fragments that are about 50% of the size of EDIM-VP6 could be used in
vaccine formulations.

| VP6 protein fragments | % Protection from rotavirus shedding in stools |
|---|---|
| MBP::VP6_{AB} | 80 |
| MBP::VP6_{BC} | 92 |
| MBP::VP6_{CD} | >99 |

### Example 21

Chemically synthesized peptides designed from VP6 protein may be used in vaccine formulations.

To illustrate that VP6 vaccines may be formulated from synthetic peptides, a series of 12 overlapping peptides (Table 13) were designed from the carboxyl-terminal half, i.e. the CD region of EDIM VP6 protein (Example 20). The synthetic peptides were synthesized on a Perkin Elmer 9050 peptide synthesizer. The well-established solid phase method was employed utilizing orthogonally protected amino acids. Cleavage and deprotection were done in aqueous trifluoroacetic acid. These overlapping peptides contained between 18 and 31 amino acids (Table 12). Peptide #6-14, a 25 mer, contains a 14-amino acid sequence that has been identified by a proliferation assay to be an H-2^{d} CD4 epitope. This 14-mer peptide (#6-14) was also synthesized.

Six-week-old rotavirus antibody-free female BALB/c mice (Harlan Sprague) were used for vaccination. Blood and stool specimens were collected from the animals prior to vaccination. The animals were immunized intranasally with 50 μg of synthetic peptides in a 50-μl volume. The animals received two immunizations separated by a biweekly interval. The adjuvant E. coli LT(R192G) was coadministered with the test vaccine.

Four weeks after the last immunization the animals were bled and stool specimens were collected to measure antibody response. Each animal was challenged with a 100 ID₅₀ dose, which is equivalent to 4×10⁴ ffu of EDIM virus, passage 9. Two stool pellets were collected into 1.0 ml of Earle's balanced salt solution (EBSS) from each mouse for seven or more days and stored at −20° C. Rotavirus antigen was measured in the stool by EIA to determine shedding (See Example 6).

The protective efficacies of 12 peptides summarized in Table 13. The peptide that gave the best protection was Peptide #6-14 inducing 98% protection. The remaining protection levels range between 0 to 85%. The results in this example provide the evidence that chemically synthesized peptides can be used in formulating rotavirus vaccines.

TABLE 13
Protective efficacies of synthetic peptides derived from the carboxyl
half of EDIMVP6.

| | | | % |
|---|---|---|---|
| Peptide No. | Sequence | SEQ. ID. NO. | Protection |
| #1 | CAINAPANIQQFEHIVQL | SEQ. I.D. | 0 |
| | RRVLTTA | No. 15 | |
| #2 | PDAERFSFPRVINSADGA | SEQ. I.D. | 70 |
| | | No. 16 | |
| #3 | FSFPRVINSADGATTWY | SEQ. I.D. | 58 |
| | FNPVILRPNNVEV | No. 17 | |
| #4 | FNPVILRPNNVEVEFLLN | SEQ. I.D. | 77 |
| | GQVINTYQARF | No. 18 | |
| #5 | NGQVINTYQARFGTIVA | SEQ. I.D. | 0 |
| | RNFDTIRLSFQLM | No. 19 | |
| #6 | RNFDTIRLSFQLMRPPN | SEQ. I.D. | 85 |
| | MTPAVTAL | No. 20 | |
| #7 | MTPAVTALFPNAQPFEH | SEQ. I.D. | 0 |
| | HATVGLTLRIDSA | No. 21 | |
| #8 | HATVLTLRIDSAICESVL | SEQ. I.D. | 51 |
| | ADASETMLANV | No. 22 | |
| #9 | VLADASETMLANVTSVR | SEQ. I.D. | 0 |
| | QEYAI | No. 23 | |
| #10 | QEYAIPVGPVFPPGMNW | SEQ. I.D. | 0 |
| | TDLITNYSPSRED | No. 24 | |
| #11 | TDLITNYSPSREDNLQRV | SEQ. I.D. | 65 |
| | FTVASIRSMLVK | No. 25 | |
| #6-14 | RLSFQLMRPPNMTP | SEQ. ID. NO. | 98 |

### Example 22

Expression of Human Rotavirus VP6.

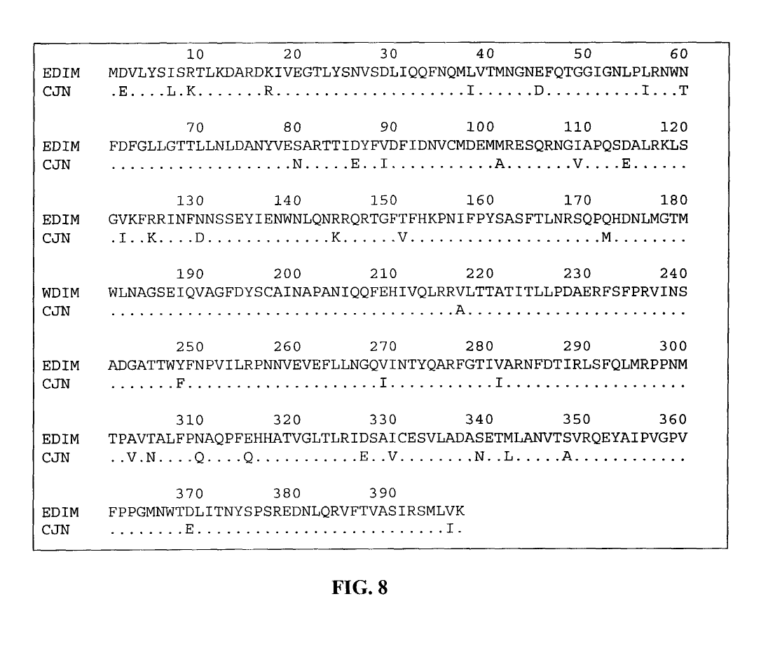

Although murine and human rotavirus VP6 proteins are highly homologous (see FIG. 8) it may be advantageous to use a human rotavirus VP6 protein for vaccination. Example 20 outlines the steps taken to clone the human rotavirus VP6. The VP6 from a human rotavirus strain CJN is cloned and its nucleotide sequence determined using standard techniques known in the art. Current Protocols in Molecular Biology, Eds. Ausubel, et al., John Wiley & Sons, Inc. In Example 20, the VP6 protein was expressed as a fusion protein for development of a vaccine candidate to be tested in mice and humans. The chimeric protein is tested in the mouse model in Example 22 to establish that a human VP6 protein from a group A virus can cross-protect against a heterologous group A (mouse EDIM) rotavirus.

### Example 23

Construction of recombinant pMAL-c2X expressing MBP::CJN-VP6::His6

To facilitate purification of a full length chimeric VP6, the plasmid pMAL-c2X (New England Biolabs, Beverly, Mass.) was used to express chimeric MBP::CJN-VP6::His6. The method for construction was essentially the same as that described in Example 2. cDNAs encoding full-length CJN VP6 were synthesized by polymerase chain reaction (PCR) using the plasmids genomic double stranded RNA as template and gene specific primers designed from the CJN sequence. The nucleotide sequence (SEQ. ID. NO. 3) was then determined by sequencing techniques commonly used in the art.

### Example 24

Expression and purification of chimeric EDIM VP6 proteins.

Recombinant bacteria obtained in Example 23 were grown as described in Example 2. MBP::CJN-VP6::His6 was expressed and purified as described in Example 2. Immunoblot analyses of the expressed chimeric VP6 showed that it could react with antibodies raised against MBP, human group A rotaviruses (DAKO, Carpinteria Calif.) and His6 (FIG. 8). Chimeric CJN-VP6 was purified using amylose resin and Talon resin. It was found that the purified preparation also contained truncated MBP::VP6 lacking the C-terminal His6 residues. Full-length MBP::VP6::His6 was then purified from the truncated proteins using Talon affinity resin containing the divalent cobalt which selectively binds His6 (BD Biosciences/Clontech, Palo Alto Calif.). To do this, the protein samples were denatured by adding guanidine-HCl, Tris-HCl (pH 8) and NaCl (final concentrations of 6M, 50 mM and 400 mM, respectively). Talon resin was washed twice with sample lysis buffer (6 M guanidine-HCl, 400 mM NaCl, 50 mM Tris-HCl, pH 8). The protein solution was added to the washed Talon resin. The mixture was then gently agitated for at least 2 hours on a platform shaker. The resin was spun down in a centrifuge (700 g, 5 min.) and the supernatant was discarded. The resin was washed by adding 10 bed-volumes of lysis buffer to the resin and the mixture was again agitated for 10 min. on a platform shaker. The resin was spun down as before (700 g, 5 min.) and the supernatant was discarded. The resin was washed in this way for a total of 4 times. The resin was resuspended in 1 bed-volume of lysis buffer and transferred to a 2 ml gravity-flow column. The resin was then washed twice with a wash buffer containing 8M urea, 400 mM NaCl, 50 mM Tris-HCl, pH 8. The bound MBP::VP6::His6 was then eluted from the resin with elution buffer (6M urea, 100 mM NaCl, 200 mM imidazole, 500 mM EDTA, 50 mM Tris-HCl, pH 8). The eluted protein was subjected to buffer exchange to PBS by using Centriprep 50 filters (Amicon Inc., Beverly Mass.).

### Example 25

Immunoblot analysis of chimeric MBP::CJN-VP6::His6

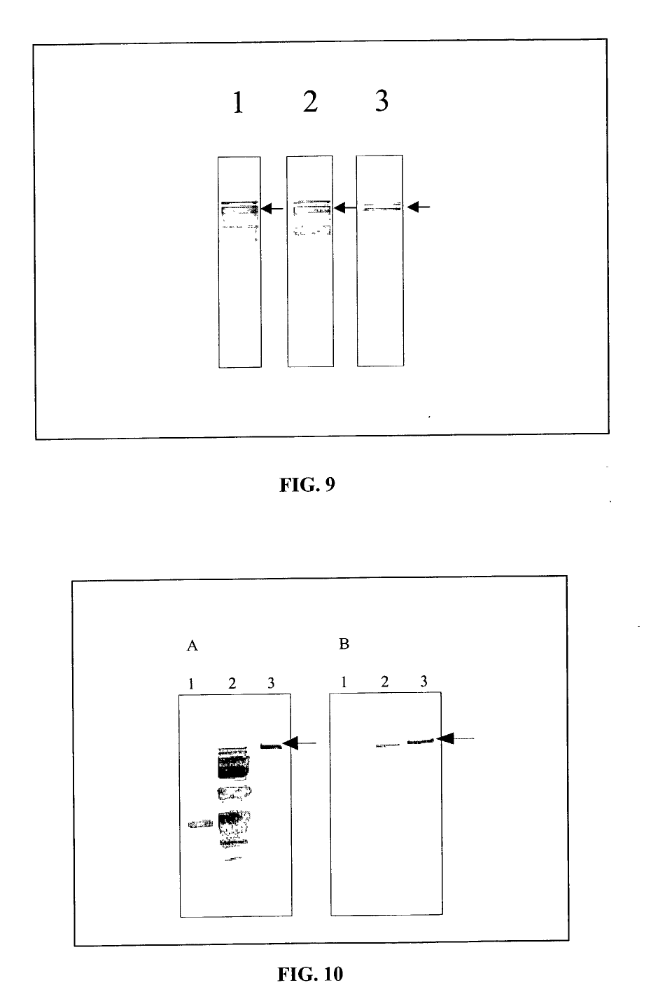

Purified MBP::VP6::His6 protein was analyzed by Immunoblot analysis to determine its purify (FIG. 9). Purified MBP::VP6::His6 protein was subjected to SDS-polyacrylamide gel electrophoresis and blotted onto a nitrocellulose sheet. The sheet was blocked with 5% skim milk in Tris-HCl buffer (TBS; 50 mM Tris-HCl, pH 7.5, 0.9% NaCl). Duplicate sheets were then incubated with anti-MBP (New England Biolabs, Inc., Beverly Mass.) or anti-His6 (Santa Cruz Biotechnology, Santa Cruz, Calif.) sera. After washing with 0.1% Tween-20 in TBS, the strips were incubated with goat anti-rabbit IgG conjugated to alkaline phosphatase (Life Technologies, Gaithersburg, Md.). The strips were washed with TBS and then incubated with 4-chloro-3-indolylphosphate and nitroblue tetrazolium (Life Technologies, Gaithersburg, Md.) to visualize bound antibodies. Preliminary experiments revealed a single protein corresponding to MBP::VP6::His6 that appeared to be free of major contamination by truncated proteins (FIG. 6).

### Example 26

Human CJN rotavirus-derived chimeric VP6 induced heterologous protection in BALB/c mice.

Almost all circulating and several newly emerging rotavirus strains are group A strains. The amino acid sequences of these group A human rotavirus VP6 proteins are highly conserved. To develop a VP6-based vaccine, the human rotavirus CJN-VP6 nucleotide sequence was cloned into the E. coli expression plasmid pMAL-c2X to express MBP::CJN-VP6. To assist in the purification of the complete chimeric VP6, a peptide containing six histidine (His6) residues was engineered into the carboxyl end of the VP6 protein to create MBP::CJN-VP6::His6.

Because mice are not susceptible to infection by human strains of rotavirus, homologous protection (e.g., protection against infection by the human CJN rotavirus in mice immunized with MBP::CJN-VP6::His6) cannot be evaluated. Because 91% of the amino acids of the CJN-VP6 protein are identical to those of EDIM-VP6 (FIG. 8), it was speculated that the human rotavirus CJN-VP6 protein would provide heterologous protection against mouse EDIM infection.

To test this hypothesis, groups of 6 to 8 BALB/c mice were immunized with either chimeric MBP::EDIM-VP6::His6 or chimeric MBP::CJN-VP6::His6. For this experiment, the mice received 2 intranasal immunizations of the chimeric CJN VP6 or EDIM VP6 proteins (9 μg) together with 10 μg LT(R192G). In addition 0.09 μg of CJN VP6 was also evaluated. Four weeks after the last immunization, the immunized group of mice and unimmunized control group were challenged with EDIM rotavirus, and VP6-specific immune responses and reduction in shedding was determined by enzyme-linked immunosorbant assay (ELISA, Example 5 and Example 6). The 0.9 μg and 0.09 μg of chimeric human CJN-VP6 preparation were found to induce approximately 98.7% and 95.4% protection against EDIM shedding respectively (Table 14). However, the immune response to the 0.09 μg was substantially lower than that of the 0.9 μg dose and the immune responses were not correlated with the level of protection induced. The results of these studies unequivocally demonstrated that a VP6 vaccine derived from the human group A rotavirus strain CJN is capable of protecting mice against infection by another group A rotavirus (i.e., mouse EDIM strain).

TABLE 14
Serum Rotavirus IgG Responses and Protection Against EDIM Shedding
after i.n. Immunization of BALB/c Mice with EDIM or CJN VP6 and
LT(R192G).

| | | | IgG titers | % protection |
|---|---|---|---|---|
| Immunogen | Dose (μg) | N | (ng/ml) | from shedding |
| None | — | 14 | >100 | 0 |
| EDIM VP6 | 9 | 8 | 31,374 | 98.7 |
| CJN VP6 | 9 | 6 | 117,789 | 86.0 |
| CJN VP6 | 0.09 | 5 | 8,113 | 95.4 |

N = number of mice in the experiment in Example 26.

### Example 27

A synthetic E. coli-codon-optimized VP6 gene derived from the human rotavirus strain CJN to increase protein expression.

To produce chimeric VP6 for use in vaccine compositions in a cost efficient manner problems associated with protein overexpression have to be overcome. Overexpression of foreign proteins in bacteria, yeast and mammalian cells sometimes lead to the synthesis of incomplete heterologous proteins. In many instances, the reason for incomplete protein synthesis has been traced to the codons in the foreign genes, which are not favorably used in the heterologous organisms. By consulting an E. coli codon usage table, the native codons, which are biased against by E. coli codon usage, in the nucleotide sequence of the human rotavirus CJN-VP6, will be altered to those that are more favorably used by E. coli cells for protein synthesis. Because of codon redundancy, one can generate a new nucleotide sequence for the CJN-VP6 gene without actually changing the amino acid sequence. It is anticipated that the codon-optimized sequence will increase the yield of complete VP6.

The method of Withers-Martinez was used to design, assemble and amplify the synthetic human rotavirus CJN-VP6 gene sequence to enhance protein expression. A computer program (http://www.entelechon.com/engibacktranslation.html, Entelechon GmbH, Regensburg, Germany) was used to back-translate the CJN protein sequence into an E. coli-codon-optimized CJN-VP6 nucleotide sequence.

Two sets of oligonucleotides (SEQ. ID. NO. 24-79) derived from the optimized CJN-VP6 sequence were designed. One set of oligonucleotides encompasses the sense strands of the synthetic gene (SEQ. ID. NO. 24-51) while the second set encompasses the antisense strand (SEQ. ID. NO. 52-79). The oligonucleotide containing the stop codon has 6 histidine codons preceding it (SEQ. ID. NO. 52). The synthetic CJN-VP6 gene contains only the His6 tag and the fusion tag MBP for purification of the protein.

The two sets of nucleotides are used in the polymerase chain reaction (PCR) to assemble and amplify the entire synthetic CJN-VP6::His6 gene. The PCR product is then purified using a PCR purification kit (Qiagen, Valencia, Calif.), and cloned into an E. coli expression plasmid using the procedure described in Example 1.

### Example 28

Expression of synthetic CJN-VP6::6×His vaccine protein.

Recombinant MBP::CJN-VP6::His6 was expressed from the plasmid containing the synthetic VP6 gene in BL21 strain of E. coli. The method used for induction of protein expression was performed as described in Example 2.

The immunoblot in FIG. 12 shows that the quantity of truncated chimeric VP6 was greatly reduced while the amount of full-length chimeric VP6 was expressed in higher quantities by the codon-optimized, synthetic gene. The amount of protein expressed was quantified using quantitative immunoblots which utilizes known quantities of pure maltose-binding protein for the determination of the amount of full-length chimeric VP6. It was found that the yield was 36 mg per liter of cells which was 18 times the amount of the protein expressed from the plasmid harboring the unmodified, native VP6 gene in the same bacterial strain.

### Example 29

Enhanced expression of MBP::CJN-VP6::VP6.

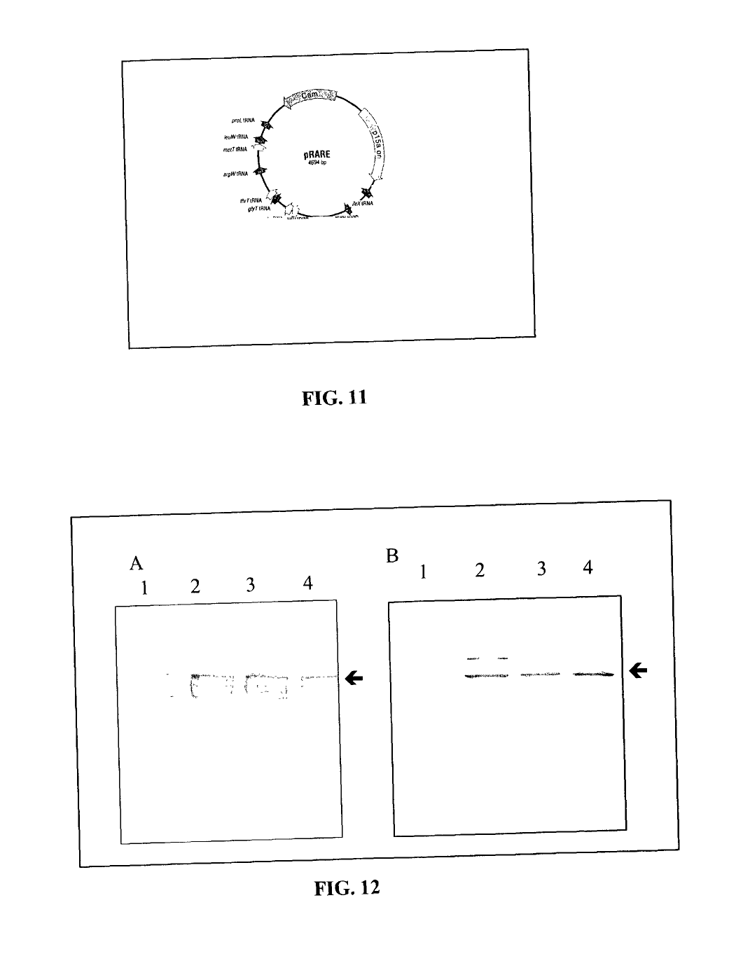

Another method to produce VP6 proteins in a cost efficient manner is to express the chimeric CJN VP6 in Rosetta cell strains that contain a plasmid called pRARE (FIG. 11). This plasmid supplies tRNA of rare codons that could enhance expressing of the unmodified, native CJN VP6 gene. Rosetta cells were transformed with a plasmid containing the unmodified, native CJN VP6 gene constructed by the procedure already described in Example 1.

FIG. 12 shows the results of immunoblot analyses and clearly demonstrates that VP6 was expressed in higher quantities by the unmodified, native CJN sequence. Quantitative immunnoblot analysis using purified maltose-binding proteins as standard showed that full-length CJN VP6 was expressed at 42 mg per liter of culture and is about 21 times the amount of VP6 expressed in BL21 cells that does not containing the pRARE plasmid.

### Example 30

Microcapsules containing the present invention for vaccine delivery.

This example relates to the method of delivering the present invention in microcapsules formulated with the adjuvant Adjumer (Parallel Solutions, Inc.). We have already provided evidence to show that this adjuvant enabled the present invention to induce 80% protection against rotavirus infection (see Example 12).

One application of microcapsules is to administer the present invention by, but not limited to, mucosal immunization. In this example, the oral route is the a priori route for delivery. This is because the effectiveness of some oral vaccines has been shown to increase significantly by incorporation of vaccines into microcapsules, which are known to selectively enter antigen-sampling microfold (M) cells.

The gastrointestinal tract provides a variety of morphological (e.g., epithelial cells, mucus) and physiological (e.g., enzymes, pH, bile salts) barriers to the soluble vaccine antigens. To overcome these barriers, microcapsules for delivery have been designed to target a specialized cell population, the microfold (M) cells. M cells are follicle-associated epithelial cells that are specialized for transcytosis of microorganisms and particles to the underlying organized mucosal lymphoid tissues. M cells contain a special lymphoid pocket into which transcytosed particles are released. These pockets are enriched with lymphocytes and are apart of the mucosal inductive sites. The effectiveness of oral vaccines has been shown to increase significantly by incorporation of vaccines into microcapsules. Formulations comprising recombinant CJN VP6 of the present invention incorporated into Adjumer microcapsules for the purpose of increasing protection levels (i.e., >80%), may be prepared using the methods described by Andrianov, A K. (Biomaterials. 19:109-115, 1998), Payne, L G. (Vaccine. 16:92-98, 1998), and Andrianov, A K and Payne, L G. (Adv Drug Deliv Rev. 34:155-170, 1998). Typically, 64 ml of 0.2% (w/v) solution of Adjumer is mixed with 0.25 ml of phosphate buffered salt containing 500 μg of protein (recombinant VP6), to which 118 ml of 6.2% (w/v) of sodium chloride solution in water is added. The mixture is shaken and incubated at room temperature for 6 min. The suspension is poured into 101 ml of 8.8% (w/v) calcium chloride solution in water. The suspension is stirred using a magnetic stirrer for 20 min. Microspheres are isolated by centrifugation, washed with deionized water and finally collected by centrifugation. To determine the quantities of microcapsules required for delivering the desired vaccine doses, the microspheres are heated in boiling water for 5 min. The amount of released protein is compared to known quantities of recombinant VP6 protein following electrophoresis in polyacrylamide gel containing SDS.

To test the efficacy of these vaccines, mice are orally immunized, according to the protocol described in Example 4, with two empirically determined doses (e.g., 10 and 50 μg) of nonencapsulated or capsulated vaccine. The efficacy of the vaccines and the immune responses of the microparticulate formulations may be determined using the methods described in Examples 5, 6, and 7 above.

### Example 31

Human Vaccine Trial

A statistically significant number of volunteers is enrolled in a study to test the safety and efficacy of the full-length, peptide, or chimeric rotavirus fusion protein vaccine compositions of the present invention. A geographical location or locations for the test is selected on the basis that the area is known to have been the site of past rotavirus outbreaks. The ratio of vaccine to placebo groups is randomized to result in a range from at least 1:1 to no more than 2:1 ratio within the group. This randomization is designed to provide appropriately large groups for statistical analysis of the efficacy of the vaccine.

The vaccine composition used in this study contains the chimeric rotavirus fusion protein comprising VP6 and MBP, the full-length VP6 alone or in combination with a further rotavirus protein, or single or multiple peptide vaccines. The vaccine composition consists of a sufficiently high concentration of rotavirus protein so as to be effective to induce a protective immune response when the composition is administered parenterally or mucosally. Parenteral administration is via intramuscular injection, preferably via needle-free methods. In both cases any of the adjuvants which are disclosed in the specification may be used.

The chimeric fusion protein is prepared according to the Examples described above. The placebo consists of an equal volume of buffered saline and is to be given mucosally or parenterally. Vaccine and placebo are supplied as individual doses that are stored at −20° C. and thawed immediately prior to use.

To determine the amount of vaccine necessary, different concentrations may be administered experimentally to a mouse. An effective concentration can be extrapolated and a comparable amount used in human subjects,

Blood samples are collected from all of the subjects for use in various laboratory assays. For example, enzyme immunoassay may be performed to evaluate the extent of the immune response elicited in each of the vaccinated individuals in response to the vaccine or placebo administered. Such techniques are well known in the art.

At the time of vaccination with either the test vaccine or the placebo, individuals participating in this study are healthy. Test subjects are assigned to receive vaccine or placebo in a double-blind fashion using a block randomization scheme. An appropriate number of doses are administered over a given period of time, e.g., two months, to elicit an immune response.

Study participants are monitored throughout the following year to determine the incidence of rotavirus infection and the subsequent development of disease conditions. Participating subjects are contacted on a periodic basis during this period to inquire about symptoms of rotaviral disease, both in the test subject and in the subject's community. Local epidemiological surveillance records may also be accessed.

The results of the above described study are assessed using standard statistical methods. The vaccine is well tolerated at the effective dose. The epidemic curves of outbreaks of rotavirus in the geographic areas tested are assessed and the distribution of episodes of rotaviral disease are established. The incidence of rotavirus caused disease in immunized individuals is reduced to a statistically significant extent as compared to those individuals receiving the placebo.

What is claimed is:

1. An isolated and purified recombinant human rotavirus VP6 polypeptide comprising an amino acid sequence having at least 98% amino acid sequence identity to SEQ. ID. NO. 4.

2. The isolated and purified recombinant human rotavirus VP6 polypeptide of claim 1, wherein the amino acid sequence has at least 99% amino acid sequence identity to SEQ. ID. NO.4.

3. The isolated and purified recombinant human rotavirus VP6 polypeptide of claim 1, wherein the amino acid sequence comprises SEQ. ID. NO. 4.

4. An immunogenic composition comprising a recombinant human rotavirus VP6 polypeptide comprising an amino acid sequence having at least 98% identity to SEQ. ID. NO. 4 and a pharmaceutical carrier.

5. The composition of claim 4 further comprising an adjuvant, wherein said adjuvant is effective in stimulating a disease-reducing immunogenic response to the recombinant human rotavirus YP6 polypeptide.

6. The composition of claim 4, wherein the amino acid sequence has at least 99% sequence identity to SEQ. ID. NO. 4.

7. The composition of claim 4, wherein the amino acid sequence comprises SEQ. ID. NO.4.

8. The composition of claim 4, wherein the recombinant human rotavirus VP6 protein is in chemical association with a protein partner.

9. The composition of claim 8, wherein the recombinant human rotavirus VP6 protein and the protein partner are chemically conjugated.

10. The composition of claim 8, wherein the recombinant human rotavirus VP6 protein and the protein partner are expressed as a fusion protein.

11. The composition of claim 7, wherein the protein partner does not interfere with expression of the recombinant human rotavirus VP6 polypeptide, the protein partner prevents complex formation by the recombinant human rotavirus VP6 polypeptide, and the protein partner facilitates purification of said recombinant rotavirus fusion protein.

12. The composition of claim 8, wherein the protein partner is selected from the group consisting of maltose binding protein, poly-histidine residues, S-Tag, glutathione-S-transferase, thioredoxin, β-galactosidase, nonapeptide epitope tag from influenza hemagglutinin, a 11 -amino acid epitope tag from vesicular stomatitis virus, a 12-amino acid epitope from the heavy chain of human Protein C, green fluorescent protein, cholera holotoxin, cholera A subunit, cholera A1 subunit, cholera A2 subunit, cholera B subunit, labile holotoxin, labile toxin subunit A, labile toxin subunit B, streptavidin, dihydrofolate reductase, and mixtures thereof.

13. The composition of claim 4, wherein the pharmaceutical carrier is suitable for parenteral administration.

14. The composition of claim 4, wherein the pharmaceutical carrier is suitable for intranasal administration.

15. The composition of claim 4, wherein the pharmaceutical carrier is suitable for oral administration.

16. The composition of claim 4, wherein the pharmaceutical carrier comprises a microencapsulated VP6 protein.

17. The composition of claim 5, wherein said adjuvant is selected from the group consisting of cholera holotoxin, cholera subunit A1, cholera subunit A2, cholera subunit B, heat labile holotoxin, heat labile subunit A, heat labile subunit B, PCPP, QS-21, QS-7, CTA1-DD, CpG DNA, and dsRNA.

18. A recombinant rotavirus fusion protein composition comprising:
a) a recombinant human CJN rotavirus VP6 protein;
b) a fusion protein partner in genetic association with said human CJN rotavirus VP6 protein;
c) an adjuvant; and
d) a pharmaceutical carrier;
wherein said adjuvant is effective in stimulating a disease-reducing immunogenic response to said rotavirus fusion protein.

19. The composition of claim 18, wherein said fusion protein partner is in genetic association with said rotavirus subunit protein, wherein said fusion protein partner does not interfere with expression of said rotavirus subunit protein, said fusion protein partner prevents complex formation by said rotavirus subunit protein, and said fusion protein partner facilitates purification of said recombinant rotavirus fusion protein.

20. The composition of claim 18, wherein said fusion protein partner is selected from the group consisting of maltose binding protein, poly-histidine residues, S-Tag, glutathione-S-transferase, thioredoxin, β-galactosidase, nonapeptide epitope tag from influenza hemagglutinin, a 11-amino acid epitope tag from vesicular stomatitis virus, a 12-amino acid epitope from the heavy chain of human Protein C, green fluorescent protein, cholera holotoxin or its A1, A2, A or B subunit, labile holotoxin or its A1, A2, A or B subunit, streptavidin and dihydrofolate reductase.

21. The composition of claim 18, wherein said adjuvant is selected from the group consisting of cholera holotoxin, cholera subunit Al, cholera subunit B, heat labile holotoxin, heat labile subunit A, heat labile subunit B, PCPP, QS-21, QS-7, CTA1-DD, CpG DNA, and dsRNA.
